(19) United States
(12) Patent Application Publication
(10) Pub. No.: US 2026/0124126 A1
(43) Pub. Date: May 7, 2026

(54) BIOACTIVE GLASS CONTAINING DENTIFRICE FORMULATIONS

(71) Applicant: CORNING INCORPORATED, CORNING, NY (US)

(72) Inventors: Qiang Fu, Painted Post, NY (US); Nicholas Michael Stone-Weiss, CORNING, NY (US)

(21) Appl. No.: 19/122,866

(22) PCT Filed: Nov. 2, 2023
(86) PCT No.: PCT/US2023/036663

§ 371 Date: Apr. 21, 2025

Related U.S. Application Data
(60) Provisional application No. 63/423,906, filed on Nov. 9, 2022.

(51) Int. Cl. A61K 8/28; A61K 8/19; A61K 8/22; A61K 8/24; A61K 8/25; A61K 8/26; A61K 8/27; A61K 8/29; A61K 8/34; A61Q 11/00; C03C 4/00; C03C 12/00

(52) U.S. Cl. CPC A61K 8/28; A61K 8/19; A61K 8/22; A61K 8/24; A61K 8/25; A61K 8/26; A61K 8/27; A61K 8/29; A61K 8/345; A61Q 11/00; C03C 4/0021; C03C 12/00; A61K 2800/28

(57) ABSTRACT

A dentifrice formulation includes a solvent, a humectant, and a bioactive glass. The bioactive glass includes greater than or equal to 15 wt % and less than or equal to 45 wt % SiO₂, greater than or equal to 30 wt % and less than or equal to 60 wt % CaO, greater than or equal to 8 wt % and less than or equal to 30 wt % P₂O₅, and greater than or equal to 2 wt % and less than or equal to 15 wt % ZrO₂.

### CROSS-REFERENCE TO RELATED APPLICATIONS

This application claims the benefit of priority under 35 U.S.C. § 119 of U.S. Provisional Application No. 63/423,906 filed Nov. 9, 2022, the content of which is incorporated herein by reference in its entirety.

### BACKGROUND

### Field

The present specification generally relates to bioactive glass and, more specifically, to dentifrice formulations containing the bioactive glass.

### Technical Background

Oral diseases pose a major health burden worldwide and affect nearly 3.5 billion people throughout their lifetime, causing pain, discomfort, disfigurement, and even death. Globally, it is estimated that 2.3 billion people suffer from caries of permanent teeth and more than 530 million children suffer from caries of primary teeth. The dissolution of apatite crystals and the net loss of calcium, phosphate and other ions from the tooth (demineralization) accounts for the formation of dental caries. The caries can be managed non-invasively through a remineralization process, in which calcium and phosphate ions are supplied from an external source to the tooth to promote crystal deposition into voids in demineralized enamel. Calcium phosphate phases in both crystalline (brushite, β-tricalcium phosphate, octocalcium phosphate, hydroxyapatite, fluorapatite and enamel apatite) and amorphous forms have been used in the remineralization process. Among these materials, amorphous calcium phosphate (bioactive glass) has shown the most promising evidence in the remineralization process. There is a strong desire to develop new bioactive glass compositions that can promote the remineralization process to prevent or repair tooth caries.

### SUMMARY

According to embodiments, a dentifrice formulation comprises: a solvent; a humectant; and a bioactive glass, wherein the bioactive glass comprises: greater than or equal to 15 wt % and less than or equal to 45 wt % SiO₂, greater than or equal to 30 wt % and less than or equal to 60 wt % CaO, greater than or equal to 8 wt % and less than or equal to 30 wt % P₂O₅, and greater than or equal to 2 wt % and less than or equal to 15 wt % ZrO₂.

According to one or more embodiments, a dentifrice formulation comprises a solvent; a humectant; and a bioactive glass, wherein the bioactive glass comprises: greater than or equal to 20.0 wt % and less than or equal to 39.5 wt % SiO₂; greater than or equal to 0.0 wt % and less than or equal to 14.5 wt % MgO; greater than or equal to 32.5 wt % and less than or equal to 47.0 wt % CaO; greater than or equal to 0.0 wt % and less than or equal to 14.5 wt % SrO; greater than or equal to 0.0 wt % and less than or equal to 14.5 wt % ZnO; greater than or equal to 9.5 wt % and less than or equal to 24.5 wt % P₂O₅; and greater than or equal to 4.0 wt % and less than or equal to 10.0 wt % ZrO₂.

According to one or more embodiments, a dentifrice formulation comprises: greater than or equal to 2 wt % and less than or equal to 15 wt % bioactive glass; greater than or equal to 30 wt % and less than or equal to 45 wt % solvent; greater than or equal to 40 wt % and less than or equal to 50 wt % humectant; greater than or equal to 0.2 wt % and less than or equal to 1.0 wt % fluoride ions; greater than or equal to 0.5 wt % and less than or equal to 5.0 wt % flavoring; greater than or equal to 0.5 wt % and less than or equal to 1.5 wt % surfactant; greater than or equal to 0.1 wt % and less than or equal to 0.5 wt % sweetener; greater than or equal to 2 wt % and less than or equal to 8 wt % abrasives; and greater than or equal to 0.1 wt % and less than or equal to 0.5 wt % whitening agents.

Additional features and advantages will be set forth in the detailed description, which follows, and in part will be readily apparent to those skilled in the art from that description or recognized by practicing the embodiments described herein, including the detailed description, which follows, the claims, as well as the appended drawings.

It is to be understood that both the foregoing general description and the following detailed description describe various embodiments and are intended to provide an overview or framework for understanding the nature and character of the claimed subject matter. The accompanying drawings are included to provide a further understanding of the various embodiments, and are incorporated into and constitute a part of this specification. The drawings illustrate the various embodiments described herein, and together with the description serve to explain the principles and operations of the claimed subject matter.

### BRIEF DESCRIPTION OF THE DRAWINGS

FIG. 1 graphically depicts weight loss versus time of bioactive glass compositions according to embodiments disclosed and described herein and conventional glass compositions in water;

FIG. 2 graphically depicts weight loss versus time of bioactive glass compositions according to embodiments disclosed and described herein and conventional glass compositions in artificial saliva;

FIG. 3 is scanning electron microscope (SEM) images of bioactive glass compositions according to embodiments disclosed and described herein before and after exposure to artificial saliva;

FIG. 4 is SEM images of conventional glass compositions before and after exposure to artificial saliva;

FIG. 5 is a graph showing x-ray diffraction (XRD) data of bioactive glass compositions according to embodiments disclosed and described herein and conventional glass compositions after exposure to artificial saliva;

FIG. 6 is a graph showing XRD data of bioactive glass compositions according to embodiments disclosed and described herein before and after exposure to artificial saliva;

FIG. 7 is a graph showing XRD data of conventional glass compositions before and after exposure to artificial saliva;

FIG. 8 is a graph showing XRD data of bioactive glass compositions according to embodiments disclosed and described herein before and after exposure to artificial saliva;

FIG. 9 is a graph showing XRD data of bioactive glass compositions according to embodiments disclosed and described herein before and after exposure to artificial saliva;

FIG. 10 is a graph showing XRD data of conventional glass compositions before and after exposure to artificial saliva;

FIG. 11 is a graph showing pH versus time of bioactive glass compositions according to embodiments disclosed and described herein in artificial saliva and water;

FIG. 12 is a graph showing pH versus time of conventional glass compositions in artificial saliva and water;

FIG. 13 is a graph showing normalized loss versus time of bioactive glass compositions according to embodiments disclosed and described herein and conventional glass compositions in artificial saliva and water;

FIG. 14 is a graph showing normalized loss versus time of bioactive glass compositions according to embodiments disclosed and described herein and conventional glass compositions in artificial saliva and water;

FIG. 15 is a bar graph of silicon normalized loss of bioactive glass compositions according to embodiments disclosed and described herein and conventional glass compositions after exposure to artificial saliva and water; and

FIG. 16 is a bar graph showing the results of ISO 719 testing on bioactive glass compositions according to embodiments disclosed and described herein and conventional glass compositions.

### DETAILED DESCRIPTION

Reference will now be made in detail to embodiments of bioactive glasses and dentifrice formulations containing the bioactive glass. Whenever possible, the same reference numerals will be used throughout the drawings to refer to the same or like parts. In embodiments, a dentifrice formulation comprises: a solvent; a humectant; and a bioactive glass, wherein the bioactive glass comprises: greater than or equal to 15 wt % and less than or equal to 45 wt % SiO₂, greater than or equal to 30 wt % and less than or equal to 60 wt % CaO, greater than or equal to 8 wt % and less than or equal to 30 wt % P₂O₅, and greater than or equal to 2 wt % and less than or equal to 15 wt % ZrO₂.

Bioactive glasses have widely recognized capabilities to bond strongly with hard and soft tissues, and to foster the growth of bone and cartilage cells. So far, the majority of bioactive glass compositions fall within Na₂O—CaO—SiO₂ family. A commercially available bioactive glass in this family, 45S5 Bioglass®, was patented in U.S. Pat. No. 4,234,972. The fast degradation and conversion to apatite makes these alkali-containing bioactive glasses attractive for a wide range of applications. However, the low chemical durability of the traditional bioactive glasses can be problematic when a long shelf lifetime is required in an aqueous environment. For example, a non-aqueous formula has to be developed to use 45S5 glass particulates in a dentifrice product as disclosed in U.S. Pat. No. 8,715,625. Alkali-free bioactive glass compositions in CaO—SiO₂—P₂O₅ system are disclosed in U.S. Pat. No. 5,074,916, but are not as bioactive as alkali-containing compositions. Accordingly, there is a strong demand of new dentifrice formulations, either aqueous or non-aqueous, that contains bioactive glass for regular uses.

Herein, aqueous and non-aqueous dentifrice formulations are provided that contain bioactive glass as an active ingredient. The bioactive glass disclosed and described herein demonstrates significantly improved water durability compared to the conventional 45S5 Bioglass®, making it possible for the creation of aqueous dentifrice formulations.

Bioactive glass compositions will now be described in more detail. The bioactive glass compositions disclosed and described herein have lower alkali contents that demonstrate improved water durability in aqueous solution and faster conversion to apatite in artificial saliva, compared to a traditional 45S5 glass.

Embodiments of bioactive glasses disclosed and described herein comprise silica (SiO₂) in amounts greater than or equal to 15 weight percent (wt %) and less than or equal to 45 wt %, calcium oxide (CaO) in amounts greater than or equal to 30 wt % and less than or equal to 60 wt %, phosphorus pentoxide (P₂O₅) in amounts greater than or equal to 8 wt % and less than or equal to 30 wt %, and zirconia (ZrO₂) in amounts greater than or equal to 2 wt % and less than or equal to 15 wt %. The bioactive glass compositions may also optionally include magnesium oxide (MgO) in amount less than or equal to 25 wt %, strontium oxide (SrO) in amounts less than or equal to 10 wt %, zinc oxide (ZnO) in amounts less than or equal to 10 wt %, boron trioxide (B₂O₃) in amounts less than or equal to 5 wt %, alumina (Al₂O₃) in amounts less than or equal to 5 wt %, lithia (Li₂O) in amounts less than or equal to 10 wt %, sodium oxide (Na₂O) in amounts less than or equal to 10 wt %, potassium oxide (K₂O) in amounts less than or equal to 10 wt %, and fluoride (F⁻) in amounts less than or equal to 5 wt %.

A low SiO₂ content, relative to other bioactive glass compositions, is believed to improve the bioactivity of the bioactive glass compositions disclosed and described herein. SiO₂, an oxide involved in the formation of glass, can also function to stabilize the networking structure of glasses and glass-ceramics. The amount of SiO₂ may be limited to control the melting temperature of the glass, as the melting temperature of pure SiO₂ or high-SiO₂ glasses is undesirably high. In embodiments, the bioactive glass composition comprises greater than or equal to 15 wt % and less than or equal to 45 wt % SiO₂, such as greater than or equal to 20 wt % and less than or equal to 45 wt % SiO₂, greater than or equal to 25 wt % and less than or equal to 45 wt % SiO₂, greater than or equal to 30 wt % and less than or equal to 45 wt % SiO₂, greater than or equal to 35 wt % and less than or equal to 45 wt % SiO₂, greater than or equal to 40 wt % and less than or equal to 45 wt % SiO₂, greater than or equal to 15 wt % and less than or equal to 40 wt % SiO₂, greater than or equal to 20 wt % and less than or equal to 40 wt % SiO₂, greater than or equal to 25 wt % and less than or equal to 40 wt % SiO₂, greater than or equal to 30 wt % and less than or equal to 40 wt % SiO₂, greater than or equal to 35 wt % and less than or equal to 40 wt % SiO₂, greater than or equal to 15 wt % and less than or equal to 35 wt % SiO₂, greater than or equal to 20 wt % and less than or equal to 35 wt % SiO₂, greater than or equal to 25 wt % and less than or equal to 35 wt % SiO₂, greater than or equal to 30 wt % and less than or equal to 35 wt % SiO₂, greater than or equal to 15 wt % and less than or equal to 30 wt % SiO₂, greater than or equal to 20 wt % and less than or equal to 30 wt % SiO₂, greater than or equal to 25 wt % and less than or equal to 30 wt % SiO₂, greater than or equal to 15 wt % and less than or equal to 25 wt % SiO₂, greater than or equal to 20 wt % and less than or equal to 25 wt % SiO₂, or greater than or equal to 15 wt % and less than or equal to 20 wt % SiO₂.

A high CaO content and low SiO₂ relative to other bioactive glass compositions are believed to improve the bioactivity of the bioactive glass compositions disclosed and described herein. CaO also lowers the viscosity of a glass, which enhances the formability, the strain point and the Young's modulus. However, when too much CaO is added to the bioactive glass composition, the density and the CTE of the bioactive glass composition increase. In embodiments, the bioactive glass composition comprises greater than or equal to 30 wt % and less than or equal to 60 wt % CaO, such as greater than or equal to 35 wt % and less than or equal to 60 wt % CaO, greater than or equal to 40 wt % and less than or equal to 60 wt % CaO, greater than or equal to 45 wt % and less than or equal to 60 wt % CaO, greater than or equal to 50 wt % and less than or equal to 60 wt % CaO, greater than or equal to 55 wt % and less than or equal to 60 wt % CaO, greater than or equal to 30 wt % and less than or equal to 55 wt % CaO, greater than or equal to 35 wt % and less than or equal to 55 wt % CaO, greater than or equal to 40 wt % and less than or equal to 55 wt % CaO, greater than or equal to 45 wt % and less than or equal to 55 wt % CaO, greater than or equal to 50 wt % and less than or equal to 55 wt % CaO, greater than or equal to 30 wt % and less than or equal to 50 wt % CaO, greater than or equal to 35 wt % and less than or equal to 50 wt % CaO, greater than or equal to 40 wt % and less than or equal to 50 wt % CaO, greater than or equal to 45 wt % and less than or equal to 50 wt % CaO, greater than or equal to 30 wt % and less than or equal to 45 wt % CaO, greater than or equal to 35 wt % and less than or equal to 45 wt % CaO, greater than or equal to 40 wt % and less than or equal to 45 wt % CaO, greater than or equal to 30 wt % and less than or equal to 40 wt % CaO, greater than or equal to 35 wt % and less than or equal to 40 wt % CaO, or greater than or equal to 30 wt % and less than or equal to 35 wt % CaO.

A high P₂O₅ content in combination with a high CaO content and low SiO₂ relative to other bioactive glass compositions are believed to improve the bioactivity of the bioactive glass compositions disclosed and described herein. P₂O₅ may also lower the viscosity of a glass, which enhances the formability, the strain point and the Young's modulus. However, when too much P₂O₅ is added to the bioactive glass composition, the density and the CTE of the bioactive glass composition increase. In embodiments, the bioactive glass composition comprises greater than or equal to 8 wt % and less than or equal to 30 wt % P₂O₅, such as greater than or equal to 10 wt % and less than or equal to 30 wt % P₂O₅, greater than or equal to 15 wt % and less than or equal to 30 wt % P₂O₅, greater than or equal to 20 wt % and less than or equal to 30 wt % P₂O₅, greater than or equal to 25 wt % and less than or equal to 30 wt % P₂O₅, greater than or equal to 8 wt % and less than or equal to 25 wt % P₂O₅, greater than or equal to 10 wt % and less than or equal to 25 wt % P₂O₅, greater than or equal to 15 wt % and less than or equal to 25 wt % P₂O₅, greater than or equal to 20 wt % and less than or equal to 25 wt % P₂O₅, greater than or equal to 8 wt % and less than or equal to 20 wt % P₂O₅, greater than or equal to 10 wt % and less than or equal to 20 wt % P₂O₅, greater than or equal to 15 wt % and less than or equal to 20 wt % P₂O₅, greater than or equal to 8 wt % and less than or equal to 15 wt % P₂O₅, greater than or equal to 10 wt % and less than or equal to 15 wt % P₂O₅, or greater than or equal to 8 wt % and less than or equal to 10 wt % P₂O₅.

Inclusion of ZrO₂ in the bioactive glass composition may also improve the bioactivity of the bioactive glass composition, and can improve the stability of the bioactive glass composition. However, including too much ZrO₂ can cause undesirable crystallization of the bioactive glass composition or increased viscosity of the bioactive glass compositions. In embodiments, the bioactive glass composition comprises greater than or equal to 2 wt % and less than or equal to 15 wt % ZrO₂, such as greater than or equal to 4 wt % and less than or equal to 15 wt % ZrO₂, greater than or equal to 6 wt % and less than or equal to 15 wt % ZrO₂, greater than or equal to 8 wt % and less than or equal to 15 wt % ZrO₂, greater than or equal to 10 wt % and less than or equal to 15 wt % ZrO₂, greater than or equal to 12 wt % and less than or equal to 15 wt % ZrO₂, greater than or equal to 2 wt % and less than or equal to 12 wt % ZrO₂, greater than or equal to 4 wt % and less than or equal to 12 wt % ZrO₂, greater than or equal to 6 wt % and less than or equal to 12 wt % ZrO₂, greater than or equal to 8 wt % and less than or equal to 12 wt % ZrO₂, greater than or equal to 10 wt % and less than or equal to 12 wt % ZrO₂, greater than or equal to 2 wt % and less than or equal to 10 wt % ZrO₂, greater than or equal to 4 wt % and less than or equal to 10 wt % ZrO₂, greater than or equal to 6 wt % and less than or equal to 10 wt % ZrO₂, greater than or equal to 8 wt % and less than or equal to 10 wt % ZrO₂, greater than or equal to 2 wt % and less than or equal to 8 wt % ZrO₂, greater than or equal to 4 wt % and less than or equal to 8 wt % ZrO₂, greater than or equal to 6 wt % and less than or equal to 8 wt % ZrO₂, greater than or equal to 2 wt % and less than or equal to 6 wt % ZrO₂, greater than or equal to 4 wt % and less than or equal to 6 wt % ZrO₂, greater than or equal to 2 wt % and less than or equal to 4 wt % ZrO₂.

MgO lowers the viscosity of a glass, which enhances the formability, the strain point and the Young's modulus. However, when too much MgO is added to the bioactive glass composition, the density and the CTE of the bioactive glass composition increase. In embodiments, the bioactive glass composition comprises less than or equal to 35 wt % MgO, such as greater than or equal to 0 wt % and less than or equal to 25 wt % MgO, greater than or equal to 2 wt % and less than or equal to 25 wt % MgO, greater than or equal to 5 wt % and less than or equal to 25 wt % MgO, greater than or equal to 8 wt % and less than or equal to 25 wt % MgO, greater than or equal to 10 wt % and less than or equal to 25 wt % MgO, greater than or equal to 14 wt % and less than or equal to 25 wt % MgO, greater than or equal to 18 wt % and less than or equal to 25 wt % MgO, greater than or equal to 20 wt % and less than or equal to 25 wt % MgO, greater than or equal to 0 wt % and less than or equal to 20 wt % MgO, greater than or equal to 2 wt % and less than or equal to 20 wt % MgO, greater than or equal to 5 wt % and less than or equal to 20 wt % MgO, greater than or equal to 8 wt % and less than or equal to 20 wt % MgO, greater than or equal to 10 wt % and less than or equal to 20 wt % MgO, greater than or equal to 14 wt % and less than or equal to 20 wt % MgO, greater than or equal to 18 wt % and less than or equal to 20 wt % MgO, greater than or equal to 0 wt % and less than or equal to 18 wt % MgO, greater than or equal to 2 wt % and less than or equal to 18 wt % MgO, greater than or equal to 5 wt % and less than or equal to 18 wt % MgO, greater than or equal to 8 wt % and less than or equal to 18 wt % MgO, greater than or equal to 10 wt % and less than or equal to 18 wt % MgO, greater than or equal to 14 wt % and less than or equal to 18 wt % MgO, greater than or equal to 0 wt % and less than or equal to 14 wt % MgO, greater than or equal to 2 wt % and less than or equal to 14 wt % MgO, greater than or equal to 5 wt % and less than or equal to 14 wt % MgO, greater than or equal to 8 wt % and less than or equal to 14 wt % MgO, greater than or equal to 10 wt % and less than or equal to 14 wt % MgO, greater than or equal to 0 wt % and less than or equal to 10 wt % MgO, greater than or equal to 2 wt % and less than or equal to 10 wt % MgO, greater than or equal to 5 wt % and less than or equal to 10 wt % MgO, greater than or equal to 8 wt % and less than or equal to 10 wt % MgO, greater than or equal to 0 wt % and less than or equal to 8 wt % MgO, greater than or equal to 2 wt % and less than or equal to 8 wt % MgO, greater than or equal to 5 wt % and less than or equal to 8 wt % MgO, greater than or equal to 0 wt % and less than or equal to 5 wt % MgO, greater than or equal to 2 wt % and less than or equal to 5 wt % MgO, or greater than or equal to 0 wt % and less than or equal to 2 wt % MgO.

SrO lowers the viscosity of a glass, which enhances the formability, the strain point and the Young's modulus. However, when too much SrO is added to the bioactive glass composition, the density and the CTE of the bioactive glass composition increase. In embodiments, the bioactive glass composition comprises less than or equal to 10 wt % SrO, such as greater than or equal to 0 wt % and less than or equal to 10 wt % SrO, greater than or equal to 2 wt % and less than or equal to 10 wt % SrO, greater than or equal to 4 wt % and less than or equal to 10 wt % SrO, greater than or equal to 6 wt % and less than or equal to 10 wt % SrO, greater than or equal to 8 wt % and less than or equal to 10 wt % SrO, greater than or equal to 0 wt % and less than or equal to 8 wt % SrO, greater than or equal to 2 wt % and less than or equal to 8 wt % SrO, greater than or equal to 4 wt % and less than or equal to 8 wt % SrO, greater than or equal to 6 wt % and less than or equal to 8 wt % SrO, greater than or equal to 0 wt % and less than or equal to 6 wt % SrO, greater than or equal to 2 wt % and less than or equal to 6 wt % SrO, greater than or equal to 4 wt % and less than or equal to 6 wt % SrO, greater than or equal to 0 wt % and less than or equal to 4 wt % SrO, greater than or equal to 2 wt % and less than or equal to 4 wt % SrO, or greater than or equal to 0 wt % and less than or equal to 2 wt % SrO.

ZnO lowers the viscosity of a glass, which enhances the formability, the strain point and the Young's modulus. However, when too much ZnO is added to the bioactive glass composition, the density and the CTE of the bioactive glass composition increase. In embodiments, the bioactive glass composition comprises less than or equal to 10 wt % ZnO, such as greater than or equal to 0 wt % and less than or equal to 10 wt % ZnO, greater than or equal to 2 wt % and less than or equal to 10 wt % ZnO, greater than or equal to 4 wt % and less than or equal to 10 wt % ZnO, greater than or equal to 6 wt % and less than or equal to 10 wt % ZnO, greater than or equal to 8 wt % and less than or equal to 10 wt % ZnO, greater than or equal to 0 wt % and less than or equal to 8 wt % ZnO, greater than or equal to 2 wt % and less than or equal to 8 wt % ZnO, greater than or equal to 4 wt % and less than or equal to 8 wt % ZnO, greater than or equal to 6 wt % and less than or equal to 8 wt % ZnO, greater than or equal to 0 wt % and less than or equal to 6 wt % ZnO, greater than or equal to 2 wt % and less than or equal to 6 wt % ZnO, greater than or equal to 4 wt % and less than or equal to 6 wt % ZnO, greater than or equal to 0 wt % and less than or equal to 4 wt % ZnO, greater than or equal to 2 wt % and less than or equal to 4 wt % ZnO, or greater than or equal to 0 wt % and less than or equal to 2 wt % ZnO.

According to embodiments, the bioactive glass composition may also comprise alkali metal such as Li₂O, Na₂O, and K₂O, for example. However, the alkali metal content in the bioactive glass composition should be relatively low to allow improved water durability in aqueous solutions and faster conversion to apatite in saliva. Accordingly, some embodiments of the bioactive glass composition disclosed and described herein do not include alkali metals, and other embodiments include low amounts of alkali metals.

In embodiments, the bioactive glass composition comprises less than or equal to 10 wt % Li₂O, such as greater than or equal to 0 wt % and less than or equal to 10 wt % Li₂O, greater than or equal to 2 wt % and less than or equal to 10 wt % Li₂O, greater than or equal to 4 wt % and less than or equal to 10 wt % Li₂O, greater than or equal to 6 wt % and less than or equal to 10 wt % Li₂O, greater than or equal to 8 wt % and less than or equal to 10 wt % Li₂O, greater than or equal to 0 wt % and less than or equal to 8 wt % Li₂O, greater than or equal to 2 wt % and less than or equal to 8 wt % Li₂O, greater than or equal to 4 wt % and less than or equal to 8 wt % Li₂O, greater than or equal to 6 wt % and less than or equal to 8 wt % Li₂O, greater than or equal to 0 wt % and less than or equal to 6 wt % Li₂O, greater than or equal to 2 wt % and less than or equal to 6 wt % Li₂O, greater than or equal to 4 wt % and less than or equal to 6 wt % Li₂O, greater than or equal to 0 wt % and less than or equal to 4 wt % Li₂O, greater than or equal to 2 wt % and less than or equal to 4 wt % Li₂O, or greater than or equal to 0 wt % and less than or equal to 2 wt % Li₂O.

In embodiments, the bioactive glass composition comprises less than or equal to 10 wt % Na₂O, such as greater than or equal to 0 wt % and less than or equal to 10 wt % Na₂O, greater than or equal to 2 wt % and less than or equal to 10 wt % Na₂O, greater than or equal to 4 wt % and less than or equal to 10 wt % Na₂O, greater than or equal to 6 wt % and less than or equal to 10 wt % Na₂O, greater than or equal to 8 wt % and less than or equal to 10 wt % Na₂O, greater than or equal to 0 wt % and less than or equal to 8 wt % Na₂O, greater than or equal to 2 wt % and less than or equal to 8 wt % Na₂O, greater than or equal to 4 wt % and less than or equal to 8 wt % Na₂O, greater than or equal to 6 wt % and less than or equal to 8 wt % Na₂O, greater than or equal to 0 wt % and less than or equal to 6 wt % Na₂O, greater than or equal to 2 wt % and less than or equal to 6 wt % Na₂O, greater than or equal to 4 wt % and less than or equal to 6 wt % Na₂O, greater than or equal to 0 wt % and less than or equal to 4 wt % Na₂O, greater than or equal to 2 wt % and less than or equal to 4 wt % Na₂O, or greater than or equal to 0 wt % and less than or equal to 2 wt % Na₂O.

In embodiments, the bioactive glass composition comprises less than or equal to 10 wt % K₂O, such as greater than or equal to 0 wt % and less than or equal to 10 wt % K₂O, greater than or equal to 2 wt % and less than or equal to 10 wt % K₂O, greater than or equal to 4 wt % and less than or equal to 10 wt % K₂O, greater than or equal to 6 wt % and less than or equal to 10 wt % K₂O, greater than or equal to 8 wt % and less than or equal to 10 wt % K₂O, greater than or equal to 0 wt % and less than or equal to 8 wt % K₂O, greater than or equal to 2 wt % and less than or equal to 8 wt % K₂O, greater than or equal to 4 wt % and less than or equal to 8 wt % K₂O, greater than or equal to 6 wt % and less than or equal to 8 wt % K₂O, greater than or equal to 0 wt % and less than or equal to 6 wt % K₂O, greater than or equal to 2 wt % and less than or equal to 6 wt % K₂O, greater than or equal to 4 wt % and less than or equal to 6 wt % K₂O, greater than or equal to 0 wt % and less than or equal to 4 wt % K₂O, greater than or equal to 2 wt % and less than or equal to 4 wt % K₂O, or greater than or equal to 0 wt % and less than or equal to 2 wt % K₂O.

Al₂O₃ may serve as a glass network former, similar to SiO₂. Al₂O₃ may increase the viscosity of the bioactive glass composition due to its tetrahedral coordination in a glass melt formed from a properly designed bioactive glass composition, decreasing the formability of the bioactive glass composition when the amount of Al₂O₃ is too high. In embodiments, the bioactive glass composition comprises less than or equal to 5 wt % Al₂O₃, such as greater than or equal to 0 wt % and less than or equal to 5 wt % Al₂O₃, greater than or equal to 1 wt % and less than or equal to 5 wt % Al₂O₃, greater than or equal to 2 wt % and less than or equal to 5 wt % Al₂O₃, greater than or equal to 3 wt % and less than or equal to 5 wt % Al₂O₃, greater than or equal to 4 wt % and less than or equal to 5 wt % Al₂O₃, greater than or equal to 0 wt % and less than or equal to 4 wt % Al₂O₃, greater than or equal to 1 wt % and less than or equal to 4 wt % Al₂O₃, greater than or equal to 2 wt % and less than or equal to 4 wt % Al₂O₃, greater than or equal to 3 wt % and less than or equal to 4 wt % Al₂O₃, greater than or equal to 0 wt % and less than or equal to 3 wt % Al₂O₃, greater than or equal to 1 wt % and less than or equal to 3 wt % Al₂O₃, greater than or equal to 2 wt % and less than or equal to 3 wt % Al₂O₃, greater than or equal to 0 wt % and less than or equal to 2 wt % Al₂O₃, greater than or equal to 1 wt % and less than or equal to 2 wt % Al₂O₃, or greater than or equal to 0 wt % and less than or equal to 1 wt % Al₂O₃.

In embodiments, the bioactive glass composition may include less than 5 wt % F⁻, such as greater than or equal to 0 wt % and less than or equal to 5 wt % F⁻, greater than or equal to 1 wt % and less than or equal to 5 wt % F⁻, greater than or equal to 2 wt % and less than or equal to 5 wt % F⁻, greater than or equal to 3 wt % and less than or equal to 5 wt % F⁻, greater than or equal to 4 wt % and less than or equal to 5 wt % F⁻, greater than or equal to 0 wt % and less than or equal to 4 wt % F⁻, greater than or equal to 1 wt % and less than or equal to 4 wt % F⁻, greater than or equal to 2 wt % and less than or equal to 4 wt % F⁻, greater than or equal to 3 wt % and less than or equal to 4 wt % F⁻, greater than or equal to 0 wt % and less than or equal to 3 wt % F⁻, greater than or equal to 1 wt % and less than or equal to 3 wt % F⁻, greater than or equal to 2 wt % and less than or equal to 3 wt % F⁻, greater than or equal to 0 wt % and less than or equal to 2 wt % F⁻, greater than or equal to 1 wt % and less than or equal to 2 wt % F⁻, or greater than or equal to 0 wt % and less than or equal to 1 wt % F⁻.

In one or more embodiments, the bioactive glass composition comprises greater than or equal to 20.0 wt % and less than or equal to 39.5 wt % SiO₂, greater than or equal to 0.0 wt % and less than or equal to 14.5 wt % MgO, greater than or equal to 32.5 wt % and less than or equal to 47.0 wt % CaO, greater than or equal to 0.0 wt % and less than or equal to 14.5 wt % SrO, greater than or equal to 0.0 wt % and less than or equal to 14.5 wt % ZnO, greater than or equal to 9.5 wt % and less than or equal to 24.5 wt % P₂O₅, and greater than or equal to 4.0 wt % and less than or equal to 10.0 wt % ZrO₂.

In one or more embodiments, the bioactive glass composition comprises greater than or equal to 20.5 wt % and less than or equal to 37.5 wt % SiO₂, greater than or equal to 0.0 wt % and less than or equal to 4.5 wt % MgO, greater than or equal to 32.5 wt % and less than or equal to 42.5 wt % CaO, greater than or equal to 11.5 wt % and less than or equal to 24.5 wt % P₂O₅, and greater than or equal to 4.0 wt % and less than or equal to 10.0 wt % ZrO₂.

In one or more embodiments, the bioactive glass composition comprises greater than or equal to 25 wt % and less than or equal to 35 wt % SiO₂, greater than or equal to 2.0 wt % and less than or equal to 6.0 wt % MgO, greater than or equal to 35.0 wt % and less than or equal to 45.0 wt % CaO, greater than or equal to 15.0 wt % and less than or equal to 25.0 wt % P₂O₅, and greater than or equal to 2.0 wt % and less than or equal to 6.0 wt % ZrO₂.

In embodiments, the bioactive glass composition has a melting point that is less than or equal to 1400° C., such as less than or equal to 1300° C., less than or equal to 1200° C., or less than or equal to 1100° C. In one or more embodiments the bioactive glass has a melting point that is greater than or equal to 800° C. and less than or equal to 1400° C., such as greater than or equal to 900° C. and less than or equal to 1400° C., greater than or equal to 1000° C. and less than or equal to 1400° C., greater than or equal to 1100° C. and less than or equal to 1400° C., greater than or equal to 1200° C. and less than or equal to 1400° C., greater than or equal to 1300° C. and less than or equal to 1400° C., greater than or equal to 800° C. and less than or equal to 1300° C., greater than or equal to 900° C. and less than or equal to 1300° C., greater than or equal to 1000° C. and less than or equal to 1300° C., greater than or equal to 1100° C. and less than or equal to 1300° C., greater than or equal to 1200° C. and less than or equal to 1300° C., greater than or equal to 800° C. and less than or equal to 1200° C., greater than or equal to 900° C. and less than or equal to 1200° C., greater than or equal to 1000° C. and less than or equal to 1200° C., greater than or equal to 1100° C. and less than or equal to 1200° C., greater than or equal to 800° C. and less than or equal to 1100° C., greater than or equal to 900° C. and less than or equal to 1100° C., greater than or equal to 1000° C. and less than or equal to 1100° C., greater than or equal to 800° C. and less than or equal to 1000° C., greater than or equal to 900° C. and less than or equal to 1000° C., or greater than or equal to 800° C. and less than or equal to 900° C. These relatively low melting points enable the bioactive glass composition to be melted in a small commercial furnace.

As will be described in more detail below, bioactive glass compositions according to embodiments disclosed and described herein are durable in aqueous solutions. The durability was measured by weight loss of the bioactive glass compositions after exposure to deionized water for a period of time. In embodiments, the bioactive glass composition has a weight loss that is less than or equal to 5 wt % after 7 days of exposure in deionized water, such as less than or equal to 4 wt % after 7 days in deionized water, less than or equal to 3 wt % after 7 days in deionized water, less than or equal to 2 wt % after 7 days in deionized water, or less than or equal to 1 wt % after 7 days in deionized water. In some embodiments, the bioactive glass composition does not lose a measurable amount of weight (approximately 0% weight loss) after 7 days in deionized water. By having the above durability in aqueous solutions, the bioactive glass compositions can be used in aqueous dentifrice formulations, which is not the case for glass compositions conventionally used in dentifrice formulations.

In addition to durability in aqueous solutions, bioactive glass compositions according to embodiments disclosed and described herein also have durability in saliva. The durability was measured by weight loss of the bioactive glass composition after exposure to artificial saliva manufactured by Modus Laboratories for a period of time. In embodiments, the bioactive glass compositions disclosed and described herein have a weight loss that is less than or equal to 9 wt % after 7 days of exposure to artificial saliva, such as less than or equal to 8 wt % after 7 days of exposure to artificial saliva, less than or equal to 7 wt % after 7 days of exposure to artificial saliva, less than or equal to 6 wt % after 7 days of exposure to artificial saliva, less than or equal to 5 wt % after 7 days of exposure to artificial saliva, less than or equal to 4 wt % after 7 days of exposure to artificial saliva, less than or equal to 2 wt % after 7 days of exposure to artificial saliva, or less than or equal to 1 wt % after 7 days of exposure to artificial saliva. In some embodiments, the bioactive glass composition does not lose a measurable amount of weight (approximately 0% weight loss) after 7 days in artificial saliva.

Bioactive glass compositions as disclosed and described herein also have higher bioactivity than glass compositions conventionally used in dentifrice formulations. In embodiments, formation of scattered crystals occurs within 2 days of soaking in artificial saliva. The whole surface of the glass powder was covered with a well-developed crystalline phase within 4 days of soaking in artificial saliva. The crystalline phase observed is hydroxyapatite, which indicates good bioactivity. For comparison, conventional glass compositions used in dentifrice formulations do not show apatite formation after soaking in artificial saliva for 7 days.

Bioactive glass compositions according to embodiments disclosed and described herein can be used as an active ingredient in dentifrice formulations. Dentifrice formulations according to embodiments disclosed and described herein will now be described.

Dentifrice formulations of embodiments include the bioactive glass compositions disclosed and described herein, a solvent, and one or more humectants. In embodiments, the solvent is water and will be referred to as an aqueous dentifrice formulation. In other embodiments, the solvent is a non-aqueous solvent, such as glycerin, and will be referred to as a non-aqueous dentifrice formulation. In embodiments, the dentifrice formulation comprises greater than or equal to 1 wt % and less than or equal to 20 wt % bioactive glass composition, greater than or equal to 10 wt % and less than or equal to 80 wt % surfactant (aqueous or non-aqueous), and greater than or equal to 2 wt % and less than or equal to 50 wt % humectant.

Initially, and according to embodiments, bioactive glass compositions of embodiments may be formed into a fine powder or microbeads to be added to a dentifrice formulation. This may be done by any suitable method such as, for example, ball milling, jet milling, and the like. In embodiments, the bioactive glass composition may be formed into powder or microbeads having a particle size distribution D99 that is less than or equal to 10 μM, such as less than or equal to 9 μM, less than or equal to 8 μm, less than or equal to 7 μm, less than or equal to 6 μm, less than or equal to 5 μm, less than or equal to 4 am, less than or equal to 3 μm, less than or equal to 2 μm, or less than or equal to 1 am. In embodiments, the D99 particle size distribution is greater than or equal to 1 μM and less than or equal to 10 μm, such as greater than or equal to 2 μM and less than or equal to 10 am, greater than or equal to 4 μM and less than or equal to 10 μm, greater than or equal to 6 μM and less than or equal to 10 μm, greater than or equal to 8 μm and less than or equal to 10 am, greater than or equal to 1 am and less than or equal to 8 μm, greater than or equal to 2 μm and less than or equal to 8 am, greater than or equal to 4 μm and less than or equal to 8 μm, greater than or equal to 6 am and less than or equal to 8 μm, greater than or equal to 1 am and less than or equal to 6 am, greater than or equal to 2 μm and less than or equal to 6 μm, greater than or equal to 4 am and less than or equal to 6 μm, greater than or equal to 1 am and less than or equal to 4 am, greater than or equal to 2 μm and less than or equal to 4 μm, or greater than or equal to 1 am and less than or equal to 2 μm.

Dentifrice formulations of embodiments disclosed and described herein comprise greater than or equal to 1 wt % and less than or equal to 20 wt % of the bioactive glass composition disclosed and described herein, such as greater than or equal to 2 wt % and less than or equal to 20 wt %, greater than or equal to 5 wt % and less than or equal to 20 wt %, greater than or equal to 8 wt % and less than or equal to 20 wt %, greater than or equal to 10 wt % and less than or equal to 20 wt %, greater than or equal to 12 wt % and less than or equal to 20 wt %, greater than or equal to 15 wt % and less than or equal to 20 wt %, greater than or equal to 18 wt % and less than or equal to 20 wt %, greater than or equal to 1 wt % and less than or equal to 18 wt %, greater than or equal to 2 wt % and less than or equal to 18 wt %, greater than or equal to 5 wt % and less than or equal to 18 wt %, greater than or equal to 8 wt % and less than or equal to 18 wt %, greater than or equal to 10 wt % and less than or equal to 18 wt %, greater than or equal to 12 wt % and less than or equal to 18 wt %, greater than or equal to 15 wt % and less than or equal to 18 wt %, greater than or equal to 1 wt % and less than or equal to 15 wt %, greater than or equal to 2 wt % and less than or equal to 15 wt %, greater than or equal to 5 wt % and less than or equal to 15 wt %, greater than or equal to 8 wt % and less than or equal to 15 wt %, greater than or equal to 10 wt % and less than or equal to 15 wt %, greater than or equal to 12 wt % and less than or equal to 15 wt %, greater than or equal to 1 wt % and less than or equal to 12 wt %, greater than or equal to 2 wt % and less than or equal to 12 wt %, greater than or equal to 5 wt % and less than or equal to 12 wt %, greater than or equal to 8 wt % and less than or equal to 12 wt %, greater than or equal to 10 wt % and less than or equal to 12 wt %, greater than or equal to 1 wt % and less than or equal to 10 wt %, greater than or equal to 2 wt % and less than or equal to 10 wt %, greater than or equal to 5 wt % and less than or equal to 10 wt %, greater than or equal to 8 wt % and less than or equal to 10 wt %, greater than or equal to 1 wt % and less than or equal to 8 wt %, greater than or equal to 2 wt % and less than or equal to 8 wt %, greater than or equal to 5 wt % and less than or equal to 8 wt %, greater than or equal to 1 wt % and less than or equal to 5 wt %, greater than or equal to 2 wt % and less than or equal to 5 wt %, or greater than or equal to 1 wt % and less than or equal to 2 wt %. If the dentifrice formulation comprises too much of the bioactive glass, the composition will not be will not have a proper consistency for application. However, if the dentifrice formulation does not comprise enough of the bioactive glass, it will not perform well.

The dentifrice formulation according to embodiments comprises greater than or equal to 10 wt % and less than or equal to 80 wt % solvent (either aqueous or non-aqueous), such as greater than or equal to 20 wt % and less than or equal to 80 wt %, greater than or equal to 30 wt % and less than or equal to 80 wt %, greater than or equal to 40 wt % and less than or equal to 80 wt %, greater than or equal to 50 wt % and less than or equal to 80 wt %, greater than or equal to 60 wt % and less than or equal to 80 wt %, greater than or equal to 70 wt % and less than or equal to 80 wt %, greater than or equal to 10 wt % and less than or equal to 70 wt %, greater than or equal to 20 wt % and less than or equal to 70 wt %, greater than or equal to 30 wt % and less than or equal to 70 wt %, greater than or equal to 40 wt % and less than or equal to 70 wt %, greater than or equal to 50 wt % and less than or equal to 70 wt %, greater than or equal to 60 wt % and less than or equal to 70 wt %, greater than or equal to 10 wt % and less than or equal to 60 wt %, greater than or equal to 20 wt % and less than or equal to 60 wt %, greater than or equal to 30 wt % and less than or equal to 60 wt %, greater than or equal to 40 wt % and less than or equal to 60 wt %, greater than or equal to 50 wt % and less than or equal to 60 wt %, greater than or equal to 10 wt % and less than or equal to 50 wt %, greater than or equal to 20 wt % and less than or equal to 50 wt %, greater than or equal to 30 wt % and less than or equal to 50 wt %, greater than or equal to 40 wt % and less than or equal to 50 wt %, greater than or equal to 10 wt % and less than or equal to 40 wt %, greater than or equal to 20 wt % and less than or equal to 40 wt %, greater than or equal to 30 wt % and less than or equal to 40 wt %, greater than or equal to 10 wt % and less than or equal to 30 wt %, greater than or equal to 20 wt % and less than or equal to 30 wt %, or greater than or equal to 10 wt % and less than or equal to 20 wt %.

In embodiments, the dentifrice formulation comprises one or more humectants. Humectants according to embodiments include short-chained polyalcohols For example, humectants according to embodiments preferably include short-chained polyalcohols selected from the group consisting of glycerol, sorbitol, propylene glycol, and polyethylene glycol. Dentifrice formulations according to embodiments include greater than or equal to 2 wt % and less than or equal to 50 wt % humectant, such as greater than or equal to 5 wt % and less than or equal to 50 wt %, greater than or equal to 10 wt % and less than or equal to 50 wt %, greater than or equal to 20 wt % and less than or equal to 50 wt %, greater than or equal to 30 wt % and less than or equal to 50 wt %, greater than or equal to 40 wt % and less than or equal to 50 wt %, greater than or equal to 2 wt % and less than or equal to 40 wt %, greater than or equal to 5 wt % and less than or equal to 40 wt %, greater than or equal to 10 wt % and less than or equal to 40 wt %, greater than or equal to 20 wt % and less than or equal to 40 wt %, greater than or equal to 30 wt % and less than or equal to 40 wt %, greater than or equal to 2 wt % and less than or equal to 30 wt %, greater than or equal to 5 wt % and less than or equal to 30 wt %, greater than or equal to 10 wt % and less than or equal to 30 wt %, greater than or equal to 20 wt % and less than or equal to 30 wt %, greater than or equal to 2 wt % and less than or equal to 20 wt %, greater than or equal to 5 wt % and less than or equal to 20 wt %, greater than or equal to 10 wt % and less than or equal to 20 wt %, greater than or equal to 2 wt % and less than or equal to 10 wt %, greater than or equal to 5 wt % and less than or equal to 10 wt %, or greater than or equal to 2 wt % and less than or equal to 5 wt %. Humectants prevent the loss of water and provide creamy textures to the dentifrice formulation. Adding too little humectant will result in a dry an unpleasant texture. Adding too much humectant can result in an overly wet dentifrice formulation.

In one or more embodiments, the dentifrice formulation includes other additives conventionally used in dentifrice formulations. These may include surfactants, desensitizing agents including potassium salts, fluorine sources, whitening agents, tartar control agents, antibacterial agents, abrasives including silica, binders and thickening agents, detergents, adhesion agents, foam modulators, pH modifying agents, mouth-feel agents, sweeteners, flavorants, colorants, preservatives, combinations thereof, and the like. One or more of these additives can be included in the dentifrice formulation.

In embodiments, the dentifrice formulation comprises greater than or equal to 1 wt % and less than or equal to 20 wt % bioactive glass composition, greater than or equal to 10 wt % and less than or equal to 80 wt % surfactant (aqueous or non-aqueous), and greater than or equal to 2 wt % and less than or equal to 50 wt % humectant, greater than or equal to 0.01 wt % and less than or equal to 1.00 wt % fluoride ions, greater than or equal to 0.05 wt % and less than or equal to 5.00 wt % flavoring, greater than or equal to 0.1 wt % and less than or equal to 5.0 wt % surfactant, greater than or equal to 0.1 wt % and less than or equal to 5.0 wt % sweetener, greater than or equal to 0.1 wt % and less than or equal to 2.0 wt % whitening agents, and greater than or equal to 0.1 wt % and less than or equal to 20.0 wt % abrasives.

In one or more embodiments, the dentifrice formulation comprises fluoride ion sources such as alkali metal fluorides, amine fluorides, stannous fluorides. In embodiments, the fluoride ion source is selected from the group consisting of sodium fluoride, potassium fluoride, ammonium fluoride, monofluorophosphates, and mixtures thereof. The dentifrice formulation comprises an amount of fluoride ion sources so that the amount of fluoride ions in the dentifrice formulation is within a desired range. The dentifrice formulation, according to embodiments, comprises greater than or equal to 0.01 wt % and less than or equal to 1.00 wt % fluoride ions, such as greater than or equal to 0.05 wt % and less than or equal to 1.00 wt %, greater than or equal to 0.10 wt % and less than or equal to 1.00 wt %, greater than or equal to 0.25 wt % and less than or equal to 1.00 wt %, greater than or equal to 0.50 wt % and less than or equal to 1.00 wt %, greater than or equal to 0.75 wt % and less than or equal to 1.00 wt %, greater than or equal to 0.01 wt % and less than or equal to 0.75 wt %, greater than or equal to 0.05 wt % and less than or equal to 0.75 wt %, greater than or equal to 0.10 wt % and less than or equal to 0.75 wt %, greater than or equal to 0.25 wt % and less than or equal to 0.75 wt %, greater than or equal to 0.50 wt % and less than or equal to 0.75 wt, greater than or equal to 0.01 wt % and less than or equal to 0.50 wt %, greater than or equal to 0.05 wt % and less than or equal to 0.50 wt %, greater than or equal to 0.10 wt % and less than or equal to 0.50 wt %, greater than or equal to 0.25 wt % and less than or equal to 0.50 wt %, greater than or equal to 0.01 wt % and less than or equal to 0.25 wt %, greater than or equal to 0.05 wt % and less than or equal to 0.25 wt %, greater than or equal to 0.10 wt % and less than or equal to 0.25 wt %, greater than or equal to 0.01 wt % and less than or equal to 0.10 wt %, greater than or equal to 0.05 wt % and less than or equal to 0.10 wt %, or greater than or equal to 0.01 wt % and less than or equal to 0.05 wt %.

Embodiments of dentifrice formulations comprise greater than or equal to 0.05 wt % and less than or equal to 5.00 wt % flavoring, such as greater than or equal to 0.50 wt % and less than or equal to 5.00 wt %, greater than or equal to 1.00 wt % and less than or equal to 5.00 wt %, greater than or equal to 1.50 wt % and less than or equal to 5.00 wt %, greater than or equal to 2.00 wt % and less than or equal to 5.00 wt %, greater than or equal to 2.50 wt % and less than or equal to 5.00 wt %, greater than or equal to 3.00 wt % and less than or equal to 5.00 wt %, greater than or equal to 3.50 wt % and less than or equal to 5.00 wt %, greater than or equal to 4.00 wt % and less than or equal to 5.00 wt %, greater than or equal to 4.50 wt % and less than or equal to 5.00 wt %, greater than or equal to 0.05 wt % and less than or equal to 4.50 wt % flavoring, greater than or equal to 0.50 wt % and less than or equal to 4.50 wt %, greater than or equal to 1.00 wt % and less than or equal to 4.50 wt %, greater than or equal to 1.50 wt % and less than or equal to 4.50 wt %, greater than or equal to 2.00 wt % and less than or equal to 4.50 wt %, greater than or equal to 2.50 wt % and less than or equal to 4.50 wt %, greater than or equal to 3.00 wt % and less than or equal to 4.50 wt %, greater than or equal to 3.50 wt % and less than or equal to 4.50 wt %, greater than or equal to 4.00 wt % and less than or equal to 4.50 wt %, greater than or equal to 0.05 wt % and less than or equal to 4.00 wt % flavoring, greater than or equal to 0.50 wt % and less than or equal to 4.00 wt %, greater than or equal to 1.00 wt % and less than or equal to 4.00 wt %, greater than or equal to 1.50 wt % and less than or equal to 4.00 wt %, greater than or equal to 2.00 wt % and less than or equal to 4.00 wt %, greater than or equal to 2.50 wt % and less than or equal to 4.00 wt %, greater than or equal to 3.00 wt % and less than or equal to 4.00 wt %, greater than or equal to 3.50 wt % and less than or equal to 4.00 wt %, greater than or equal to 0.05 wt % and less than or equal to 3.50 wt % flavoring, greater than or equal to 0.50 wt % and less than or equal to 3.50 wt %, greater than or equal to 1.00 wt % and less than or equal to 3.50 wt %, greater than or equal to 1.50 wt % and less than or equal to 3.50 wt %, greater than or equal to 2.00 wt % and less than or equal to 3.50 wt %, greater than or equal to 2.50 wt % and less than or equal to 3.50 wt %, greater than or equal to 3.00 wt % and less than or equal to 3.50 wt %, greater than or equal to 0.05 wt % and less than or equal to 3.00 wt % flavoring, greater than or equal to 0.50 wt % and less than or equal to 3.00 wt %, greater than or equal to 1.00 wt % and less than or equal to 3.00 wt %, greater than or equal to 1.50 wt % and less than or equal to 3.00 wt %, greater than or equal to 2.00 wt % and less than or equal to 3.00 wt %, greater than or equal to 2.50 wt % and less than or equal to 3.00 wt %, greater than or equal to 0.05 wt % and less than or equal to 2.50 wt % flavoring, greater than or equal to 0.50 wt % and less than or equal to 2.50 wt %, greater than or equal to 1.00 wt % and less than or equal to 2.50 wt %, greater than or equal to 1.50 wt % and less than or equal to 2.50 wt %, greater than or equal to 2.00 wt % and less than or equal to 2.50 wt %, greater than or equal to 0.05 wt % and less than or equal to 2.00 wt % flavoring, greater than or equal to 0.50 wt % and less than or equal to 2.00 wt %, greater than or equal to 1.00 wt % and less than or equal to 2.00 wt %, greater than or equal to 1.50 wt % and less than or equal to 2.00 wt %, greater than or equal to 0.05 wt % and less than or equal to 1.50 wt % flavoring, greater than or equal to 0.50 wt % and less than or equal to 1.50 wt %, greater than or equal to 1.00 wt % and less than or equal to 1.50 wt %, greater than or equal to 0.05 wt % and less than or equal to 1.00 wt % flavoring, greater than or equal to 0.50 wt % and less than or equal to 1.00 wt %, or greater than or equal to 0.05 wt % and less than or equal to 0.50 wt % flavoring.

In embodiments, the dentifrice formulation comprises surfactant, such as surfactant selected from sodium lauryl sulfate, poloxamer (such as an ethylene oxide/propylene oxide copolymers), and mixtures thereof. The dentifrice formulation according to embodiments comprises greater than or equal to 0.1 wt % and less than or equal to 5.0 wt % surfactant, such as greater than or equal to 0.5 wt % and less than or equal to 5.0 wt %, greater than or equal to 1.0 wt % and less than or equal to 5.0 wt %, greater than or equal to 2.0 wt % and less than or equal to 5.0 wt %, greater than or equal to 3.0 wt % and less than or equal to 5.0 wt %, greater than or equal to 4.0 wt % and less than or equal to 5.0 wt %, greater than or equal to 0.1 wt % and less than or equal to 4.0 wt %, greater than or equal to 0.5 wt % and less than or equal to 4.0 wt %, greater than or equal to 1.0 wt % and less than or equal to 4.0 wt %, greater than or equal to 2.0 wt % and less than or equal to 4.0 wt %, greater than or equal to 3.0 wt % and less than or equal to 4.0 wt %, greater than or equal to 0.1 wt % and less than or equal to 3.0 wt %, greater than or equal to 0.5 wt % and less than or equal to 3.0 wt %, greater than or equal to 1.0 wt % and less than or equal to 3.0 wt %, greater than or equal to 2.0 wt % and less than or equal to 3.0 wt %, greater than or equal to 0.1 wt % and less than or equal to 2.0 wt %, greater than or equal to 0.5 wt % and less than or equal to 2.0 wt %, greater than or equal to 1.0 wt % and less than or equal to 2.0 wt %, greater than or equal to 0.1 wt % and less than or equal to 1.0 wt %, greater than or equal to 0.5 wt % and less than or equal to 1.0 wt %, or greater than or equal to 0.1 wt % and less than or equal to 0.5 wt %.

According to embodiments, dentifrice formulations comprise greater than or equal to 0.1 wt % and less than or equal to 5.0 wt % sweetener, such as greater than or equal to 0.5 wt % and less than or equal to 5.0 wt %, greater than or equal to 1.0 wt % and less than or equal to 5.0 wt %, greater than or equal to 2.0 wt % and less than or equal to 5.0 wt %, greater than or equal to 3.0 wt % and less than or equal to 5.0 wt %, greater than or equal to 4.0 wt % and less than or equal to 5.0 wt %, greater than or equal to 0.1 wt % and less than or equal to 4.0 wt %, greater than or equal to 0.5 wt % and less than or equal to 4.0 wt %, greater than or equal to 1.0 wt % and less than or equal to 4.0 wt %, greater than or equal to 2.0 wt % and less than or equal to 4.0 wt %, greater than or equal to 3.0 wt % and less than or equal to 4.0 wt %, greater than or equal to 0.1 wt % and less than or equal to 3.0 wt %, greater than or equal to 0.5 wt % and less than or equal to 3.0 wt %, greater than or equal to 1.0 wt % and less than or equal to 3.0 wt %, greater than or equal to 2.0 wt % and less than or equal to 3.0 wt %, greater than or equal to 0.1 wt % and less than or equal to 2.0 wt %, greater than or equal to 0.5 wt % and less than or equal to 2.0 wt %, greater than or equal to 1.0 wt % and less than or equal to 2.0 wt %, greater than or equal to 0.1 wt % and less than or equal to 1.0 wt %, greater than or equal to 0.5 wt % and less than or equal to 1.0 wt %, or greater than or equal to 0.1 wt % and less than or equal to 0.5 wt %.

Dentifrice formulations according to embodiments comprise whitening agents, such as whitening agents selected from the group consisting of titanium dioxide, hydrogen peroxide, sodium tripolyphosphates, and mixtures thereof. The dentifrice formulation according to embodiments comprises greater than or equal to 0.1 wt % and less than or equal to 2.0 wt % whitening agents, such as greater than or equal to 0.5 wt % and less than or equal to 2.0 wt %, greater than or equal to 1.0 wt % and less than or equal to 2.0 wt %, greater than or equal to 1.5 wt % and less than or equal to 2.0 wt %, greater than or equal to 0.1 wt % and less than or equal to 1.5 wt %, greater than or equal to 0.5 wt % and less than or equal to 1.5 wt %, greater than or equal to 1.0 wt % and less than or equal to 1.5 wt %, greater than or equal to 0.1 wt % and less than or equal to 1.0 wt %, greater than or equal to 0.5 wt % and less than or equal to 1.0 wt %, or greater than or equal to 0.1 wt % and less than or equal to 0.5 wt %.

In embodiments, dentifrice formulations comprise abrasives, such as abrasives selected from the group consisting of silica, zinc orthophosphate, sodium bicarbonate, alumina, calcium carbonate, calcium pyrophosphate, and mixtures thereof. The dentifrice formulation of embodiments comprises greater than or equal to 0.1 wt % and less than or equal to 20.0 wt % abrasives, such as greater than or equal to 1.0 wt % and less than or equal to 20.0 wt %, greater than or equal to 5.0 wt % and less than or equal to 20.0 wt %, greater than or equal to 10.0 wt % and less than or equal to 20.0 wt %, greater than or equal to 15.0 wt % and less than or equal to 20.0 wt %, greater than or equal to 0.1 wt % and less than or equal to 15.0 wt %, greater than or equal to 1.0 wt % and less than or equal to 15.0 wt %, greater than or equal to 5.0 wt % and less than or equal to 15.0 wt %, greater than or equal to 10.0 wt % and less than or equal to 15.0 wt %, greater than or equal to 0.1 wt % and less than or equal to 10.0 wt %, greater than or equal to 1.0 wt % and less than or equal to 10.0 wt %, greater than or equal to 5.0 wt % and less than or equal to 10.0 wt %, greater than or equal to 0.1 wt % and less than or equal to 5.0 wt %, greater than or equal to 1.0 wt % and less than or equal to 5.0 wt %, or greater than or equal to 0.1 wt % and less than or equal to 1.0 wt %.

In some embodiments, a dentifrice composition comprises a tartar control (anti-calculus) agent. These agents include phosphate and polyphosphate salts such as monobasic, dibasic and tribasic sodium phosphates, sodium tripolyphosphate, sodium trimetaphosphate, sodium hexametaphosphate and mixtures thereof. Embodiments of dentifrice formulations comprise greater than or equal to 0.05 wt % and less than or equal to 0.50 wt % tartar control agents, such as greater than or equal to 0.10 wt % and less than or equal to 0.50 wt %, greater than or equal to 0.20 wt % and less than or equal to 0.50 wt %, greater than or equal to 0.25 wt % and less than or equal to 0.50 wt %, greater than or equal to 0.30 wt % and less than or equal to 0.50 wt %, greater than or equal to 0.40 wt % and less than or equal to 0.50 wt %, greater than or equal to 0.05 wt % and less than or equal to 0.40 wt %, greater than or equal to 0.10 wt % and less than or equal to 0.40 wt %, greater than or equal to 0.20 wt % and less than or equal to 0.40 wt %, greater than or equal to 0.25 wt % and less than or equal to 0.40 wt %, greater than or equal to 0.30 wt % and less than or equal to 0.40 wt %, greater than or equal to 0.05 wt % and less than or equal to 0.30 wt %, greater than or equal to 0.10 wt % and less than or equal to 0.30 wt %, greater than or equal to 0.20 wt % and less than or equal to 0.30 wt %, greater than or equal to 0.25 wt % and less than or equal to 0.30 wt %, greater than or equal to 0.05 wt % and less than or equal to 0.25 wt %, greater than or equal to 0.10 wt % and less than or equal to 0.25 wt %, greater than or equal to 0.20 wt % and less than or equal to 0.25 wt %, greater than or equal to 0.05 wt % and less than or equal to 0.20 wt %, greater than or equal to 0.10 wt % and less than or equal to 0.20 wt %, or greater than or equal to 0.05 wt % and less than or equal to 0.10 wt %.

In some embodiments, the dentifrice formulation may include other active materials for the prevention or treatment of the disorders of hard or soft tissues of the oral cavity. These may include antibacterial or antistatin agents and the like. Embodiments of dentifrice formulations comprise greater than or equal to 0.05 wt % and less than or equal to 0.50 wt % of other active materials, such as greater than or equal to 0.10 wt % and less than or equal to 0.50 wt %, greater than or equal to 0.20 wt % and less than or equal to 0.50 wt %, greater than or equal to 0.25 wt % and less than or equal to 0.50 wt %, greater than or equal to 0.30 wt % and less than or equal to 0.50 wt %, greater than or equal to 0.40 wt % and less than or equal to 0.50 wt %, greater than or equal to 0.05 wt % and less than or equal to 0.40 wt %, greater than or equal to 0.10 wt % and less than or equal to 0.40 wt %, greater than or equal to 0.20 wt % and less than or equal to 0.40 wt %, greater than or equal to 0.25 wt % and less than or equal to 0.40 wt %, greater than or equal to 0.30 wt % and less than or equal to 0.40 wt %, greater than or equal to 0.05 wt % and less than or equal to 0.30 wt %, greater than or equal to 0.10 wt % and less than or equal to 0.30 wt %, greater than or equal to 0.20 wt % and less than or equal to 0.30 wt %, greater than or equal to 0.25 wt % and less than or equal to 0.30 wt %, greater than or equal to 0.05 wt % and less than or equal to 0.25 wt %, greater than or equal to 0.10 wt % and less than or equal to 0.25 wt %, greater than or equal to 0.20 wt % and less than or equal to 0.25 wt %, greater than or equal to 0.05 wt % and less than or equal to 0.20 wt %, greater than or equal to 0.10 wt % and less than or equal to 0.20 wt %, or greater than or equal to 0.05 wt % and less than or equal to 0.10 wt %.

Embodiments of aqueous dentifrice formulations comprise greater than or equal to 2 wt % and less than or equal to 15 wt % bioactive glass, greater than or equal to 30 wt % and less than or equal to 45 wt % solvent (water), greater than or equal to 40 wt % and less than or equal to 50 wt % humectant, greater than or equal to 0.2 wt % and less than or equal to 1.0 wt % fluoride ions, greater than or equal to 0.5 wt % and less than or equal to 5.0 wt % flavoring, greater than or equal to 0.5 wt % and less than or equal to 1.5 wt % surfactant, greater than or equal to 0.1 wt % and less than or equal to 0.5 wt % sweetener, greater than or equal to 2 wt % and less than or equal to 8 wt % abrasives, and greater than or equal to 0.1 wt % and less than or equal to 0.5 wt % whitening agents.

Embodiments of non-aqueous dentifrice formulation comprise greater than or equal to 2 wt % and less than or equal to 15 wt % bioactive glass, greater than or equal to 50 wt % and less than or equal to 75 wt % solvent (glycerin), greater than or equal to 15 wt % and less than or equal to 20 wt % humectant, greater than or equal to 0.2 wt % and less than or equal to 1.0 wt % fluoride ions, greater than or equal to 0.5 wt % and less than or equal to 5.0 wt % flavoring, greater than or equal to 0.5 wt % and less than or equal to 1.5 wt % surfactant, greater than or equal to 0.1 wt % and less than or equal to 0.5 wt % sweetener, greater than or equal to 2 wt % and less than or equal to 8 wt % abrasives, and greater than or equal to 0.1 wt % and less than or equal to 0.5 wt % whitening agents.

A first aspect includes a dentifrice formulation comprising: a solvent; a humectant; and a bioactive glass, wherein the bioactive glass comprises: greater than or equal to 15 wt % and less than or equal to 45 wt % SiO₂, greater than or equal to 30 wt % and less than or equal to 60 wt % CaO, greater than or equal to 8 wt % and less than or equal to 30 wt % P₂O₅, and greater than or equal to 2 wt % and less than or equal to 15 wt % ZrO₂.

A second aspect includes the dentifrice formulation of the first aspect wherein the bioactive glass comprises: greater than or equal to 20.0 wt % and less than or equal to 39.5 wt % SiO₂; greater than or equal to 0.0 wt % and less than or equal to 14.5 wt % MgO; greater than or equal to 32.5 wt % and less than or equal to 47.0 wt % CaO; greater than or equal to 0.0 wt % and less than or equal to 14.5 wt % SrO; greater than or equal to 0.0 wt % and less than or equal to 14.5 wt % ZnO; greater than or equal to 9.5 wt % and less than or equal to 24.5 wt % P₂O₅; and greater than or equal to 4.0 wt % and less than or equal to 10.0 wt % ZrO₂.

A third aspect includes the dentifrice formulation of the first or second aspect, wherein the bioactive glass has a weight loss that is less than or equal to 5 wt % after 7 days of exposure in deionized water.

A fourth aspect includes the dentifrice formulation of any of the first to third aspects, wherein the solvent is an aqueous solvent.

A fifth aspect includes the dentifrice formulation of any of the first to third aspects, wherein the solvent is a non-aqueous solvent.

A sixth aspect includes the dentifrice formulation of the fifth aspect, wherein the solvent is glycerin.

A seventh aspect includes the dentifrice formulation of any of the first to sixth aspects, wherein the dentifrice formulation comprises: greater than or equal to 1 wt % and less than or equal to 20 wt % solvent; greater than or equal to 2 wt % and less than or equal to 50 wt % humectant; and greater than or equal to 1 wt % and less than or equal to 20 wt % bioactive glass.

An eighth aspect includes the dentifrice formulation of any of the first to seventh aspects, wherein the humectant is a short-chained polyalcohol selected from the group consisting of glycerol, sorbitol, propylene glycol, and polyethylene glycol.

A ninth aspect includes the dentifrice formulation of any of the first to eighth aspects, further comprising one or more of a fluoride ion source, a flavoring, a surfactant, a sweetener, a whitening agent, and an abrasive.

A tenth aspect includes the dentifrice formulation of the ninth aspect, wherein the dentifrice formulation comprises a fluoride ion source selected from the group consisting of sodium fluoride, potassium fluoride, ammonium fluoride, monofluorophosphates, and mixtures thereof.

An eleventh aspect includes the dentifrice formulation of the tenth aspect, wherein the dentifrice formulation comprises greater than or equal to 0.01 wt % and less than or equal to 1.00 wt % of fluoride ions.

A twelfth aspect includes the dentifrice formulation of the ninth to eleventh aspects, wherein the dentifrice formulation comprises greater than or equal to 0.05 wt % and less than or equal to 5.00 wt % flavoring.

A thirteenth aspect includes the dentifrice formulation of any of the ninth to twelfth aspects, wherein the dentifrice formulation comprises a surfactant selected from the group consisting of sodium lauryl sulfate, poloxamer, and mixtures thereof.

A fourteenth aspect includes the dentifrice formulation of the thirteenth aspect, wherein the dentifrice formulation comprises greater than or equal to 0.1 wt % and less than or equal to 5.0 wt % surfactant.

A fifteenth aspect includes the dentifrice formulation of any of the ninth to fourteenth aspects, wherein the dentifrice formulation comprises greater than or equal to 0.1 wt % and less than or equal to 5.0 wt % sweetener.

A sixteenth aspect includes the dentifrice formulation of any of the ninth to fifteenth aspects, wherein the dentifrice formulation comprises a whitening agent selected from the group consisting of titanium dioxide, hydrogen peroxide, sodium tripolyphosphates, and mixtures thereof.

A seventeenth aspect includes the dentifrice formulation of the sixteenth aspect, wherein the dentifrice formulation comprises greater than or equal to 0.1 wt % and less than or equal to 2.0 wt % whitening agents.

An eighteenth aspect includes the dentifrice formulation of any of the ninth to seventeenth aspects, wherein the dentifrice formulation comprises an abrasive selected from the group consisting of silica, zinc orthophosphate, sodium bicarbonate, alumina, calcium carbonate, calcium pyrophosphate, and mixtures thereof.

A nineteenth aspect includes the dentifrice formulation of the eighteenth aspect, wherein the dentifrice formulation comprises greater than or equal to 0.1 wt % and less than or equal to 20.0 wt % abrasives.

A twentieth aspect includes the dentifrice formulation of any of the first to nineteenth aspects, wherein the dentifrice formulation comprises: greater than or equal to 2 wt % and less than or equal to 15 wt % bioactive glass; greater than or equal to 30 wt % and less than or equal to 45 wt % solvent; greater than or equal to 40 wt % and less than or equal to 50 wt % humectant; greater than or equal to 0.2 wt % and less than or equal to 1.0 wt % fluoride ions; greater than or equal to 0.5 wt % and less than or equal to 1.5 wt % flavoring; greater than or equal to 0.5 wt % and less than or equal to 1.5 wt % surfactant; greater than or equal to 0.1 wt % and less than or equal to 0.5 wt % sweetener; greater than or equal to 2 wt % and less than or equal to 8 wt % abrasives; and greater than or equal to 0.1 wt % and less than or equal to 0.5 wt % whitening agents.

### EXAMPLES

Embodiments will be further clarified by the following examples.

### Example 1

Twelve bioactive glass samples according to embodiments disclosed and described herein were prepared by conventional glass melting and forming techniques. The density, annealing point, and refractive index of the twelve samples were then compared to the conventional glass composition 45S5 Bioglass®. The compositions of 45S5 Bioglass® and the twelve samples are provided in Table 1 below. The annealing point and refractive index of the twelve samples and 45S5 Bioglass® are also shown in Table 1.

TABLE 1

| Oxides (wt %) | Comp. | 1 | 2 | 3 | 4 | 5 | 6 |
|---|---|---|---|---|---|---|---|
| SiO₂ | 45.0 | 39.5 | 39.5 | 39.5 | 39.5 | 39.5 | 39.5 |
| Na₂O | 24.5 | 0.0 | 0.0 | 0.0 | 0.0 | 0.0 | 0.0 |
| MgO | 0.0 | 0 | 4.5 | 9.5 | 14.5 | 0 | 0 |
| CaO | 24.5 | 47 | 42.5 | 37.5 | 32.5 | 32.5 | 32.5 |
| SrO | 0.0 | 0 | 0 | 0 | 0 | 14.5 | 0 |
| ZnO | 0.0 | 0 | 0 | 0 | 0 | 0 | 14.5 |
| P₂O₅ | 6.0 | 9.5 | 9.5 | 9.5 | 9.5 | 9.5 | 9.5 |
| ZrO₂ | 0.0 | 4 | 4 | 4 | 4 | 4 | 4 |
| Density | 2.71 | 3.03 | 2.96 | 2.95 | 2.99 | 3.18 | 3.15 |
| (g/cm³) | | | | | | | |
| Annealing | 514.80 | 759.1 | 743.2 | 738.3 | 757.3 | 729.2 | 687 |
| point (° C.) | | | | | | | |
| Refractive | 1.562 | 1.639 | 1.638 | 1.630 | 1.620 | 1.632 | 1.635 |
| index, n_{d} | | | | | | | |

| Oxides (wt %) | 7 | 8 | 9 | 10 | 11 | 12 |
|---|---|---|---|---|---|---|
| SiO₂ | 37.5 | 34.5 | 29.5 | 24.5 | 24.5 | 20.5 |
| MgO | 4.5 | 4.5 | 4.5 | 4.5 | 4.5 | 4.5 |
| CaO | 42.5 | 42.5 | 42.5 | 42.5 | 42.5 | 42.5 |
| P₂O₅ | 11.5 | 14.5 | 19.5 | 24.5 | 22.5 | 22.5 |
| ZrO₂ | 4.0 | 4.0 | 4.0 | 4.0 | 6 | 10 |
| Density | 2.98 | 2.99 | 3.03 | 3.12 | 3.16 | 3.26 |
| (g/cm³) | | | | | | |
| Annealing | 738.00 | 730.20 | 717.60 | 702.20 | 805.90 | 704.30 |
| point (° C.) | | | | | | |
| Refractive | 1.647 | 1.634 | 1.628 | 1.623 | 1.634 | 1.653 |
| index, n_{d} | | | | | | |

### Example 2

The bioactive glass composition of Sample 9 from Example 1 was ground into bioactive glass particles having a particle size distribution D99 that was less than 10 m. The weight of the bioactive glass particles was measured to obtain an initial weight. The bioactive glass particles were then soaked in deionized water for 7 days. The weight of the bioactive glass particles was measured after 1 day of soaking in water, after 4 days of soaking in water, and after 7 days of soaking in water. The weight of the glass particles after soaking in water for 1 day, soaking in water for 4 days, and soaking in water for 7 days was compared to the initial weight to determine the weight loss percentage on day 1, on day 4, and on day 7.

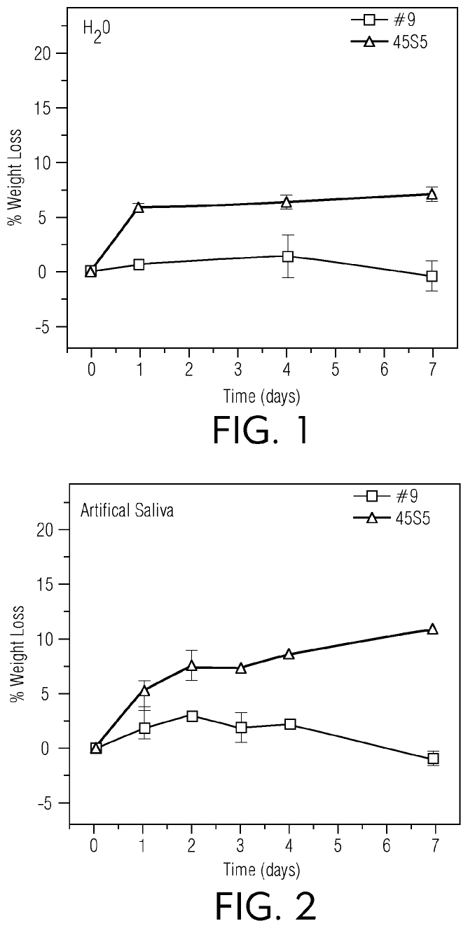

The above weight loss measurements were also performed on conventional 45S5 Bioglass®. The results of this weight loss test are provided in FIG. 1. As shown in FIG. 1, the weight loss of the bioactive glass of Sample 9 is below 2.5% and near 0% even after 7 days of soaking in water, whereas 45S5 Bioglass® has a weight loss of nearly 6% after only 1 day of soaking in water. This shows that the bioactive glass of Sample 9 has much better durability in water than 45S5 Bioglass® and, therefore, is suitable for use in aqueous dentifrice formulations.

### Example 3

The bioactive glass composition of Sample 9 from Example 1 was ground into bioactive glass particles having a particle size distribution D99 that was less than 10 m. The weight of the bioactive glass particles was measured to obtain an initial weight. The bioactive glass particles were then soaked in artificial saliva manufactured by Modus Laboratories water for 7 days. The weight of the bioactive glass particles was measured after 1 day of soaking in artificial saliva, after 4 days of soaking in artificial saliva, and after 7 days of soaking in artificial saliva. The weight of the glass particles after soaking in artificial saliva for 1 day, soaking in artificial saliva for 4 days, and soaking in artificial saliva for 7 days was compared to the initial weight to determine the weight loss percentage on day 1, on day 4, and on day 7.

The above weight loss measurements were also performed on conventional 45S5 Bioglass®. The results of this weight loss test are provided in FIG. 2. As shown in FIG. 2, the weight loss of the bioactive glass of Sample 9 is below 2.5% and near 0% even after 7 days of soaking in artificial saliva, whereas 45S5 Bioglass® has a weight loss of nearly 6% after only 1 day of soaking in artificial saliva. This shows that the bioactive glass of Sample 9 has much better durability in artificial saliva than 45S5 Bioglass® and, therefore, is better suited to non-aqueous dentifrice formulations than 45S5 Bioglass.

### Example 4

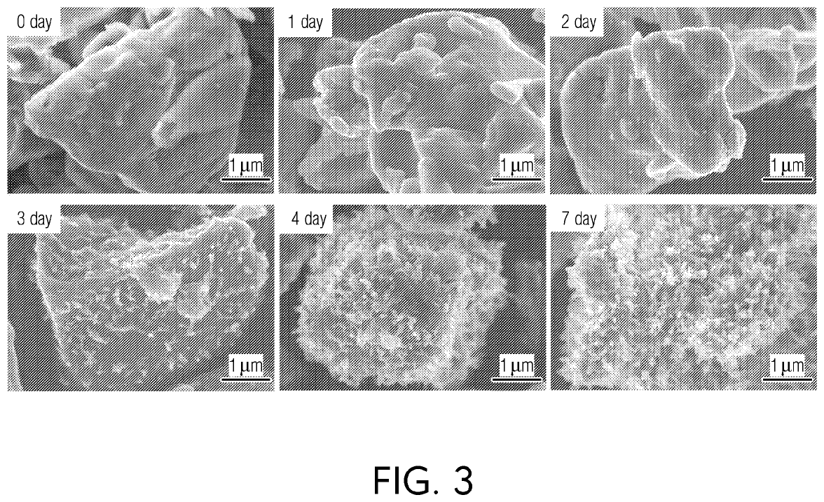

The bioactive glass composition of Sample 9 from Example 1 was ground into bioactive glass particles having a particle size distribution that was less than 10 m. The bioactive glass particles were then soaked in artificial saliva manufacture by Modus Laboratories. Scanning electron microscope (SEM) images were taken before soaking began, after 1 day of soaking in artificial saliva, after 2 days of soaking in artificial saliva, after 3 days of soaking in artificial saliva, after 4 days of soaking in artificial saliva, and after 7 days of soaking in artificial saliva. The SEM images are provided in FIG. 3. As shown in FIG. 3, within 2 days after soaking in artificial saliva, formation of scattered crystals was observed. The number and size of crystals increased with time. After 4 days, the whole surface of the bioactive glass particles was covered with a well-developed crystalline phase.

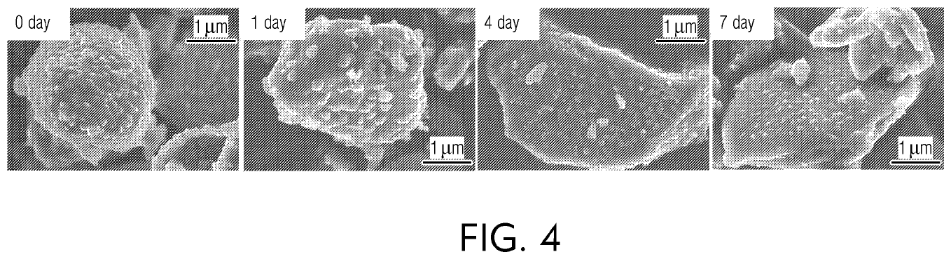

For a comparison, 45S5 Bioglass® particles were soaked in artificial saliva manufacture by Modus Laboratories. SEM images were taken before soaking began, after 1 day of soaking in artificial saliva, after 4 days of soaking in artificial saliva, and after 7 days of soaking in artificial saliva. The SEM images are provided in FIG. 4. As shown in FIG. 4, the surface morphology of 45S5 Bioglass® did not change after soaking for up to 7 days.

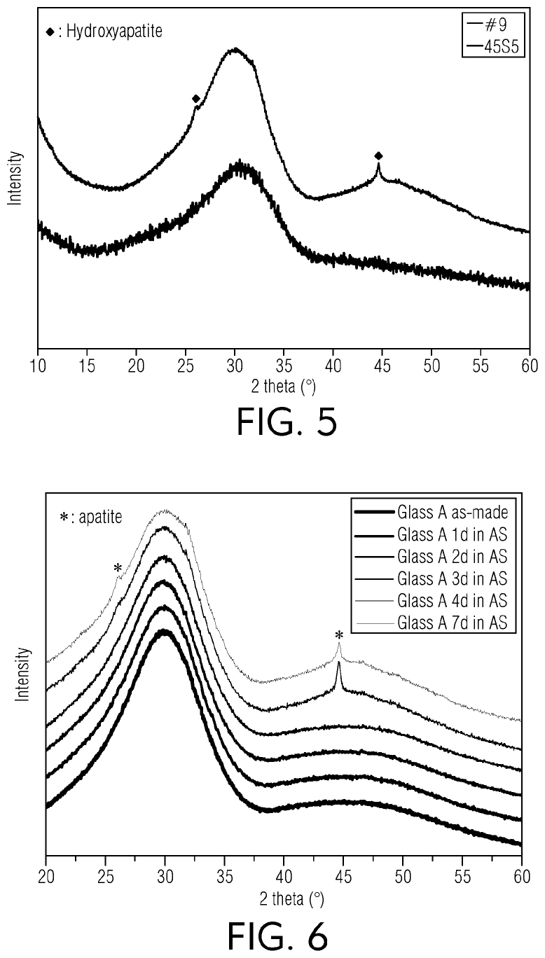

To determine the phase of the crystalline structure formed on the surface of the bioactive glass of Sample 9 from Example 1, x-ray diffraction (XRD) was conducted on the bioactive glass of Sample 9 from Example 1 after soaking in artificial saliva for 7 days. Similarly, XRD was conducted on the 45S5 Bioglass® sample after soaking in saliva for 7 days. The XRD data for both bioactive glass of Sample 9 from Example 1 and 45S5 Bioglass® are shown in FIG. 5. The XRD result in FIG. 5 show crystals formed in the bioactive glass of Sample 9 from Example 1 were hydroxyapatite while no detectable crystalline phase was observed in 45S5 Bioglass®. The bioactive glass of Sample 9 from Example 1 demonstrate a step change in bioactivity, which can be of significant advantage for cavity and hypersensitivity treatment.

In addition to the XRD data shown in FIG. 5, additional XRD data was collected on the bioactive glass of Sample 9 from Example 1 and is shown in FIG. 6. As shown in FIG. 6, six XRD plots were collected. Namely, XRD was measured on the bioactive glass of Sample 9 from Example 1 in its as-made form, after 1 day in artificial saliva, after 2 days in artificial saliva, after 3 days in artificial saliva, after 4 days in artificial saliva, and after 7 days in artificial saliva. The XRD plots in FIG. 6 show that apatite formation occurred on the bioactive glass of Sample 9 from Example 1 with 4 days of soaking in artificial saliva, and significant additional formation of apatite did not occur between days 4 and 7 of soaking in artificial saliva. This shows the increased bioactivity of the bioactive glass of Sample 9 from Example 1.

### Example 5

Additional testing was conducted to determine corrosion of the bioactive glass of Sample 9 from Example 1 compared to 45S5 Bioglass®. Initial tests were conducted by soaking the bioactive glass of Sample 9 from Example 1 and 45S5 Bioglass® in artificial saliva comprising 28 ppm Na⁺, 80 ppm K⁺, and 6 ppm Mg²⁺ for seven days. This artificial saliva had a pH of 7.1. XRD data was collected on the as-made samples, after 1 day soaking in the artificial saliva, after 4 days soaking in the artificial saliva, after 7 days soaking in the artificial saliva, after 14 days soaking in the artificial saliva, after 21 days soaking in the artificial saliva, and after 30 days soaking in the artificial saliva.

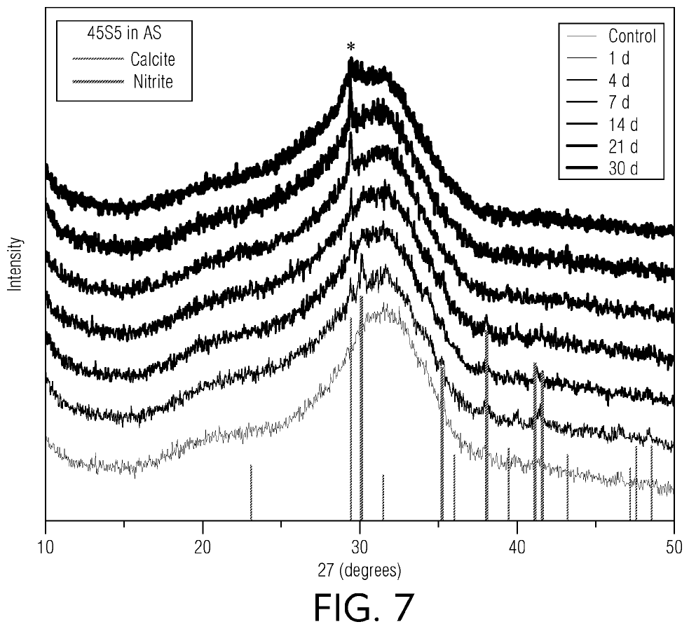

XRD data for 45S5 Bioglass® is shown in FIG. 7 and shows that natrite (Na₂CO₃) was present on the surface of the 45S5 Bioglass® even before soaking in the artificial saliva, and only non-bioactive calcite (CaCO₃) forms on the 45S5 Bioglass® after 30 days of soaking in the artificial saliva. Accordingly, 45S5 Bioglass® is not particularly bioactive.

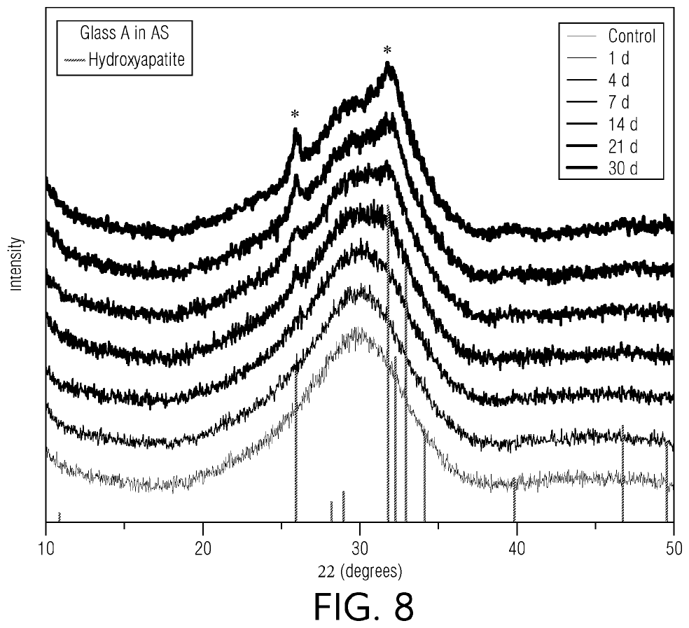

XRD data for the bioactive glass of Sample 9 from Example 1 is shown in FIG. 8 and confirms what was shown in the above examples that hydroxyapatite forms on the bioactive glass of Sample 9 from Example 1 in seven days or less.

### Example 6

Corrosion was also tested using artificial saliva manufactured by Modus Laboratories. XRD data was collected for the as-made bioactive glass of Sample 9 from Example 1 and the as-made 45S5 Bioglass®. The bioactive glass of Sample 9 from Example 1 and 45S5 Bioglass® were then soaked in the artificial saliva manufactured by Modus Laboratories for seven days and XRD data was collected on each sample after soaking.

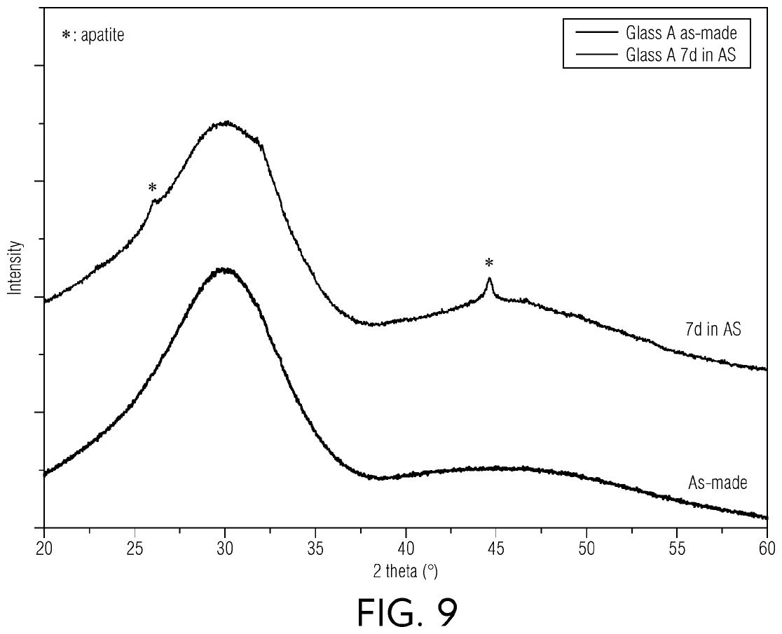

FIG. 9 shows the XRD data for the bioactive glass of Sample 9 from Example 1. This XRD data confirms what was shown in previous examples that apatite was formed within 7 days of soaking, and no nitrite to calcite was formed. Moreover, the bioactive glass of Sample 9 from Example 1 was free of surface corrosion after a year of storage.

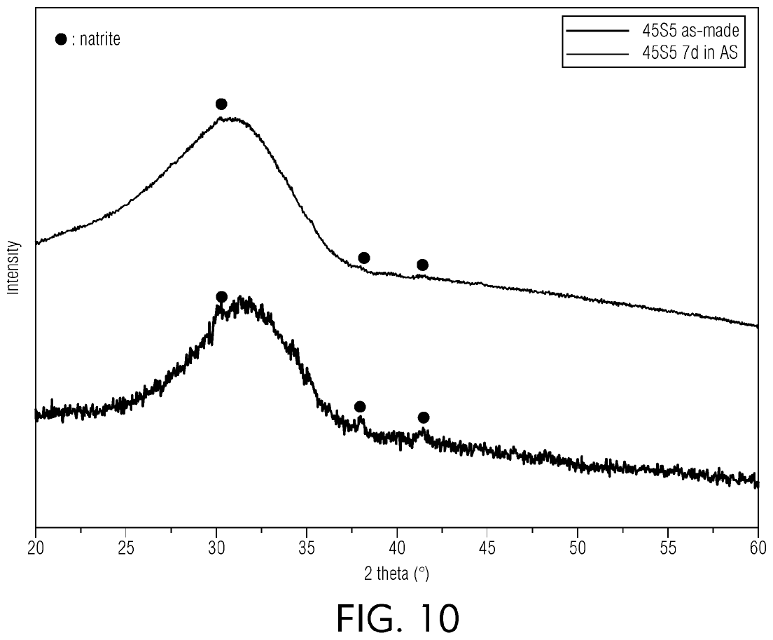

FIG. 10 is XRD data for 45S5 Bioglass® and shows that natrite was detected in the as-made 45S5 Bioglass® in only days after powder was prepared, which indicates surface corrosion. Moreover, no apatite was formed in the 45S5 Bioglass®.

### Example 7

The effect of the bioactive glass of Sample 9 from Example 1 and 45S5 Bioglass® on the pH of artificial saliva was also studied.

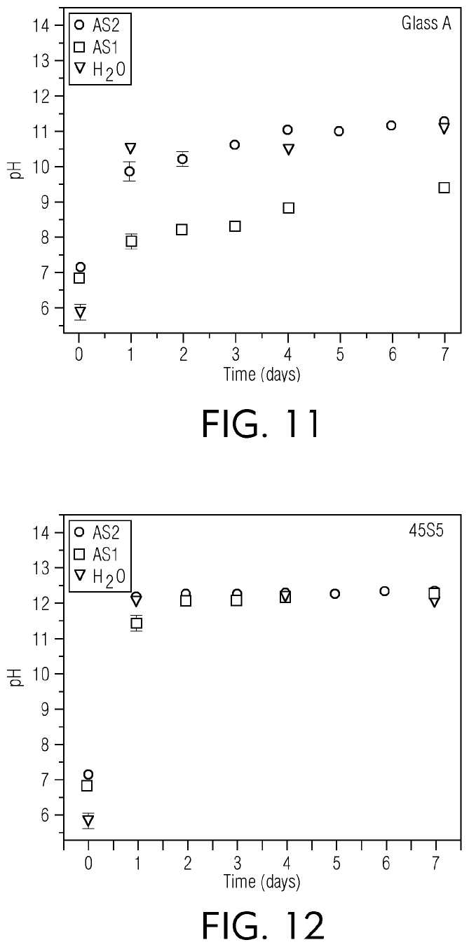

The bioactive glass of Sample 9 from Example 1 was soaked in deionized water (referred to as “H₂O”) for seven days, soaked in artificial saliva manufactured by Modus Laboratories (referred to as “AS 1”) for seven days, and soaked in artificial saliva comprising 28 ppm Na⁺, 80 ppm K⁺, and 6 ppm Mg²⁺ and having a pH of 7.1 (referred to as “AS 2”) for seven days. The pH of the H₂O, AS 1, and AS 2 were measured before the bioactive glass of Sample 9 from Example 1 (referred to as day 0 in the figures) and every day thereafter for seven days. The results of this test is shown in FIG. 11.

The 45S5 Bioglass® was soaked in H₂O for seven days, soaked in AS 1 for seven days, and soaked in AS 2 for seven days. The pH of the H₂O, AS 1, and AS 2 were measured before the 45S5 Bioglass® (referred to as day 0 in the figures) and every day thereafter for seven days. The results of this test is shown in FIG. 12.

For both the bioactive glass of Sample 9 from Example 1 and 45S5 Bioglass®, the pH of each solution (H₂O, AS 1, and AS 2) increased quickly after adding the glass. After about one day of soaking, the pH of each solution leveled off and stayed relatively consistent for the next six days. The bioactive glass of Sample 9 from Example 1 showed less of pH increase than the 45S5 Bioglass®, and particularly, the bioactive glass of Sample 9 from Example 1 showed the least pH increase in AS 1, while 45S5 Bioglass® showed a greater pH increase that bioactive glass of Sample 9 from Example 1 in each of H₂O, AS 1, and AS 2 and particularly in AS 2.

### Example 8

The normalized loss of various components in the bioactive glass of Sample 9 from Example 1 and 45S5 Bioglass® in AS 1 was also studied.

The bioactive glass of Sample 9 from Example 1 (referred to as “Glass A” in the figures) and 45S5 Bioglass® were soaked in AS 1 for seven days. The normalized loss (mg/m²) of sodium and silicon was measured for 45S5 Bioglass®, and the normalized loss of calcium and silicon was measured for bioactive glass of Sample 9 from Example 1 (the bioactive glass of Sample 9 from Example 1 does not comprise sodium).

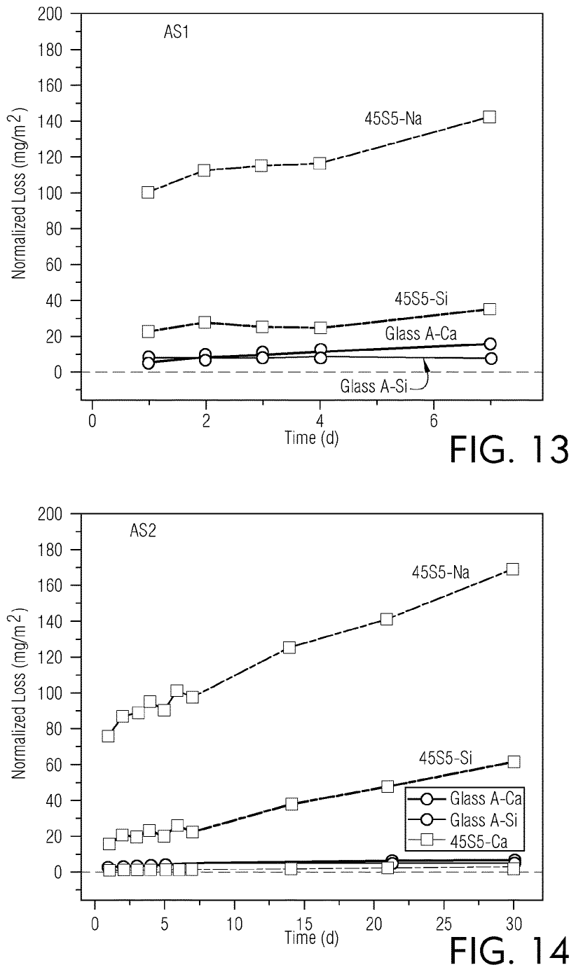

FIG. 13 shows the results of this test. As shown in FIG. 13, the bioactive glass of Sample 9 from Example 1 shows minimal normalized loss of calcium and silicon, while 45S5 Bioglass® shows much greater normalized loss of silica and extremely high normalized loss of sodium. The significant normalized loss of sodium in 45S5 Bioglass® shows that this glass dissolves much quicker than bioactive glass of Sample 9 from Example 1, and the higher normalized loss of silicon in the 45S5 Bioglass® shows that even the glass matrix is reacting in the artificial saliva. In contrast, the bioactive glass of Sample 9 from Example 1 is much less reactive.

The normalized loss was calculated by equation 1 below:

$$Normalized Loss = \frac{( {C}_{i , t} - {C}_{0} ) * V}{S S A * m * {f}_{i}} ,$$

Where C_{i,t} is the concentration of element i at time t, C₀ is the blank solution concentration, V is the solution volume, SSA is the specific surface area (measure by BET), m is the starting sample mass, and fᵢ is the mass fraction of element i.

### Example 9

The normalized loss of various components in the bioactive glass of Sample 9 from Example 1 and 45S5 Bioglass® in AS 2 was also studied.

The bioactive glass of Sample 9 from Example 1 (referred to as “Glass A” in the figures) and 45S5 Bioglass® were soaked in AS 2 for thirty days. The normalized loss (mg/m²) of sodium, silicon, and calcium was measured for 45S5 Bioglass®, and the normalized loss of calcium and silicon was measured for bioactive glass of Sample 9 from Example 1 (the bioactive glass of Sample 9 from Example 1 does not comprise sodium).

FIG. 14 shows the results of this test. As shown in FIG. 14, the bioactive glass of Sample 9 from Example 1 shows minimal normalized loss of calcium and silicon, while 45S5 Bioglass® shows much greater normalized loss of silica and extremely high normalized loss of sodium. The significant normalized loss of sodium in 45S5 Bioglass® shows that this glass dissolves much quicker than bioactive glass of Sample 9 from Example 1, and the higher normalized loss of silicon in the 45S5 Bioglass® shows that even the glass matrix is reacting in the artificial saliva. In contrast, the bioactive glass of Sample 9 from Example 1 is much less reactive.

The normalized loss of each component was calculated using Equation 1 from Example 8.

### Example 10

The normalized loss of silicon in the bioactive glass of Sample 9 from Example 1 and 45S5 Bioglass® in H₂O, AS 1, and AS 2 was further studied.

The bioactive glass of Sample 9 from Example 1 (referred to as “Glass A” in the figures) and 45S5 Bioglass® were soaked in H₂O, AS 1, and AS 2 for seven days. The normalized loss (mg/m²) of silicon was measured for the bioactive glass of Sample 9 from Example 1 and for 45S5 Bioglass®.

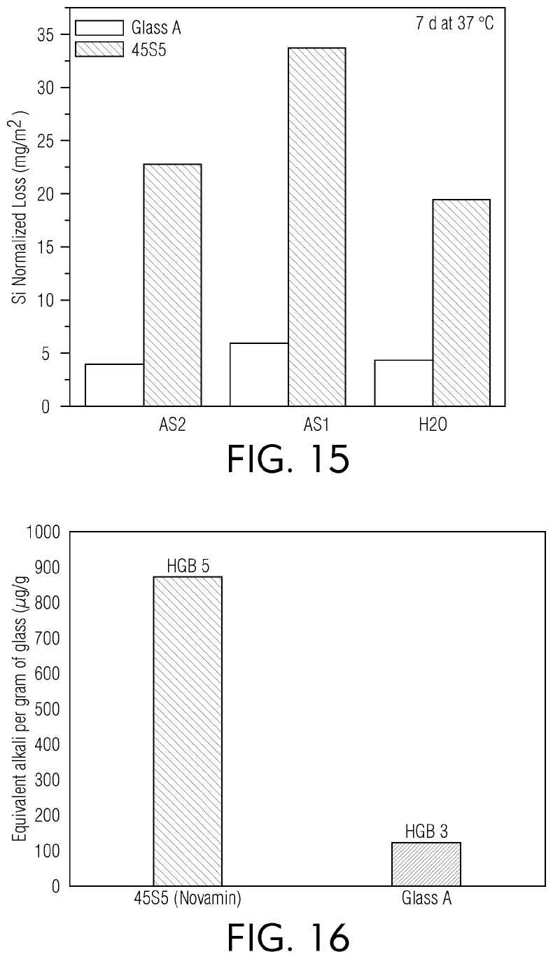

FIG. 15 shows the results of this test. As shown in FIG. 15, the bioactive glass of Sample 9 from Example 1 shows minimal normalized loss of silicon in each of H₂O, AS 1, and AS 2. In contrast, 45S5 Bioglass® shows significant normalized loss of silicon in each of H₂O, AS 1, and AS 2. The large normalized loss of silicon in 45S5 Bioglass® shows the increased reactivity of the glass and that even the glass matrix is reacting. Without being bound to any particular theory, it is believed that the high concentration of calcium in the bioactive glass of Sample 9 from Example 1 provides low reactivity.

The normalized loss of each component was calculated using Equation 1 from Example 8.

### Example 11

The water durability of the bioactive glass of Sample 9 from Example 1 and 45S5 Bioglass® was further studied.

The bioactive glass of Sample 9 from Example 1 (referred to as “Glass A” in the figures) and 45S5 Bioglass® were subjected to the ISO 719 test to determine the hydrolytic resistance of glass grains (300 μm to 425 μm) at 98° C. for 1 hour in water. FIG. 16 shows the results of this test.

As shown in FIG. 16, the bioactive glass of Sample 9 from Example 1 was rated as HGB 3 and 45S5 Bioglass® was rated as HGB 5, showing vastly increased water durability of the bioactive glass of Sample 9 from Example 1 compared to 45S5 Bioglass®.

### Example 12

Twelve aqueous dentifrice formulations were composed using the bioactive glass sample 9 from Example 1 above. The remaining components of the aqueous dentifrice formulation is provided in Table 2 below. It should be noted that glycerin is used as a humectant in the formulations in Table 2.

TABLE 2

| | 1 | 2 | 3 | 4 | 5 | 6 | 7 |
|---|---|---|---|---|---|---|---|
| Bioactive glass | 2 | 5 | 10 | 15 | 2 | 5 | 10 |
| Water | 43.6 | 40.6 | 35.6 | 30.6 | 44.38 | 41.38 | 36.38 |
| Sorbitol | 26 | 26 | 26 | 26 | 26 | 26 | 26 |
| Glycerin | 20 | 20 | 20 | 20 | 20 | 20 | 20 |
| Sodium | 1.1 | 1.1 | 1.1 | 1.1 | 0 | 0 | 0 |
| monofluorophosphate | | | | | | | |
| sodium fluoride | 0 | 0 | 0 | 0 | 0.32 | 0.32 | 0.32 |
| Stannous fluoride | 0 | 0 | 0 | 0 | 0 | 0 | 0 |
| Flavor | 1 | 1 | 1 | 1 | 1 | 1 | 1 |
| sodium lauryl sulfate | 1 | 1 | 1 | 1 | 1 | 1 | 1 |
| Sweetener | 0.2 | 0.2 | 0.2 | 0.2 | 0.2 | 0.2 | 0.2 |
| Titanium dioxide | 0.1 | 0.1 | 0.1 | 0.1 | 0.1 | 0.1 | 0.1 |
| Silica | 5 | 5 | 5 | 5 | 5 | 5 | 5 |
| Total | 100 | 100 | 100 | 100 | 100 | 100 | 100 |

| | 8 | 9 | 10 | 11 | 12 |
|---|---|---|---|---|---|
| Bioactive glass | 15 | 2 | 5 | 10 | 15 |
| Water | 31.38 | 44.24 | 41.24 | 36.24 | 31.24 |
| Sorbitol | 26 | 26 | 26 | 26 | 26 |
| Glycerin | 20 | 20 | 20 | 20 | 20 |
| Sodium | 0 | 0 | 0 | 0 | 0 |
| monofluorophosphate | | | | | |
| sodium fluoride | 0.32 | 0 | 0 | 0 | 0 |
| Stannous fluoride | 0 | 0.46 | 0.46 | 0.46 | 0.46 |
| Flavor | 1 | 1 | 1 | 1 | 1 |
| sodium lauryl sulfate | 1 | 1 | 1 | 1 | 1 |
| Sweetener | 0.2 | 0.2 | 0.2 | 0.2 | 0.2 |
| Titanium dioxide | 0.1 | 0.1 | 0.1 | 0.1 | 0.1 |
| Silica | 5 | 5 | 5 | 5 | 5 |
| Total | 100 | 100 | 100 | 100 | 100 |

Each of these dentifrice formulations had good mouth feel and stability over time. The formulations include, in part, a bioactive glass that is useful in treating or preventing various disorders of the oral cavity through enamel remineralization and caries remineralization.

### Example 13

Twelve non-aqueous dentifrice formulations were composed using the bioactive glass sample 9 from Example 1 above. The remaining components of the aqueous dentifrice formulation is provided in Table 3 below. It should be noted that glycerin is used as a solvent in the non-aqueous dentifrice formulations.

TABLE 3

| Ingredient | 1 | 2 | 3 | 4 | 5 | 6 | 7 |
|---|---|---|---|---|---|---|---|
| Bioactive glass | 2 | 5 | 10 | 15 | 2 | 5 | 10 |
| Glycerin | 69.6 | 66.6 | 61.6 | 56.6 | 70.38 | 67.38 | 62.38 |
| Poly ethylene glycol | 20 | 20 | 20 | 20 | 20 | 20 | 20 |
| Sodium | 1.1 | 1.1 | 1.1 | 1.1 | 0 | 0 | 0 |
| monofluorophosphate | | | | | | | |
| sodium fluoride | 0 | 0 | 0 | 0 | 0.32 | 0.32 | 0.32 |
| Stannous fluoride | 0 | 0 | 0 | 0 | 0 | 0 | 0 |
| Flavor | 1 | 1 | 1 | 1 | 1 | 1 | 1 |
| sodium lauryl sulfate | 1 | 1 | 1 | 1 | 1 | 1 | 1 |
| Sweetener | 0.2 | 0.2 | 0.2 | 0.2 | 0.2 | 0.2 | 0.2 |
| Titanium dioxide | 0.1 | 0.1 | 0.1 | 0.1 | 0.1 | 0.1 | 0.1 |
| Silica | 5 | 5 | 5 | 5 | 5 | 5 | 5 |
| Total | 100 | 100 | 100 | 100 | 100 | 100 | 100 |

| Ingredient | 8 | 9 | 10 | 11 | 12 |
|---|---|---|---|---|---|
| Bioactive glass | 15 | 2 | 5 | 10 | 15 |
| Glycerin | 57.38 | 70.24 | 67.24 | 62.24 | 57.24 |
| Poly ethylene glycol | 20 | 20 | 20 | 20 | 20 |
| Sodium | 0 | 0 | 0 | 0 | 0 |
| monofluorophosphate | | | | | |
| sodium fluoride | 0.32 | 0 | 0 | 0 | 0 |
| Stannous fluoride | 0 | 0.46 | 0.46 | 0.46 | 0.46 |
| Flavor | 1 | 1 | 1 | 1 | 1 |
| sodium lauryl sulfate | 1 | 1 | 1 | 1 | 1 |
| Sweetener | 0.2 | 0.2 | 0.2 | 0.2 | 0.2 |
| Titanium dioxide | 0.1 | 0.1 | 0.1 | 0.1 | 0.1 |
| Silica | 5 | 5 | 5 | 5 | 5 |
| Total | 100 | 100 | 100 | 100 | 100 |

Each of these dentifrice formulations had good mouth feel and stability over time. The formulations include, in part, a bioactive glass that is useful in treating or preventing various disorders of the oral cavity through enamel remineralization and caries remineralization.

It will be apparent to those skilled in the art that various modifications and variations can be made to the embodiments described herein without departing from the spirit and scope of the claimed subject matter. Thus, it is intended that the specification cover the modifications and variations of the various embodiments described herein provided such modification and variations come within the scope of the appended claims and their equivalents.

What is claimed is:

1. A dentifrice formulation comprising:
a solvent;
a humectant; and
a bioactive glass, wherein the bioactive glass comprises:
greater than or equal to 15 wt % and less than or equal to 45 wt % SiO₂,
greater than or equal to 30 wt % and less than or equal to 60 wt % CaO,
greater than or equal to 8 wt % and less than or equal to 30 wt % P₂O₅, and
greater than or equal to 2 wt % and less than or equal to 15 wt % ZrO₂.

2. The dentifrice formulation of claim 1, wherein the bioactive glass comprises:
greater than or equal to 20.0 wt % and less than or equal to 39.5 wt % SiO₂;
greater than or equal to 0.0 wt % and less than or equal to 14.5 wt % MgO;
greater than or equal to 32.5 wt % and less than or equal to 47.0 wt % CaO;
greater than or equal to 0.0 wt % and less than or equal to 14.5 wt % SrO;
greater than or equal to 0.0 wt % and less than or equal to 14.5 wt % ZnO;
greater than or equal to 9.5 wt % and less than or equal to 24.5 wt % P₂O₅; and
greater than or equal to 4.0 wt % and less than or equal to 10.0 wt % ZrO₂.

3. The dentifrice formulation of claim 1, wherein the bioactive glass has a weight loss that is less than or equal to 5 wt % after 7 days of exposure in deionized water.

4. The dentifrice formulation of claim 1, wherein the solvent is an aqueous solvent.

5. The dentifrice formulation of claim 1, wherein the solvent is a non-aqueous solvent.

6. The dentifrice formulation of claim 5, wherein the solvent is glycerin.

7. The dentifrice formulation of claim 1, wherein the dentifrice formulation comprises:
greater than or equal to 1 wt % and less than or equal to 20 wt % solvent;
greater than or equal to 2 wt % and less than or equal to 50 wt % humectant; and
greater than or equal to 1 wt % and less than or equal to 20 wt % bioactive glass.

8. The dentifrice formulation of claim 1, wherein the humectant is a short-chained polyalcohol selected from the group consisting of glycerol, sorbitol, propylene glycol, and polyethylene glycol.

9. The dentifrice formulation of claim 1, further comprising one or more of a fluoride ion source, a flavoring, a surfactant, a sweetener, a whitening agent, and an abrasive.

10. The dentifrice formulation of claim 9, wherein the dentifrice formulation comprises a fluoride ion source selected from the group consisting of sodium fluoride, potassium fluoride, ammonium fluoride, monofluorophosphates, and mixtures thereof.

11. The dentifrice formulation of claim 10, wherein the dentifrice formulation comprises greater than or equal to 0.01 wt % and less than or equal to 1.00 wt % of fluoride ions.

12. The dentifrice formulation of claim 9, wherein the dentifrice formulation comprises greater than or equal to 0.05 wt % and less than or equal to 5.00 wt % flavoring.

13. The dentifrice formulation of claim 9, wherein the dentifrice formulation comprises a surfactant selected from the group consisting of sodium lauryl sulfate, poloxamer, and mixtures thereof.

14. The dentifrice formulation of claim 13, wherein the dentifrice formulation comprises greater than or equal to 0.1 wt % and less than or equal to 5.0 wt % surfactant.

15. The dentifrice formulation of claim 9, wherein the dentifrice formulation comprises greater than or equal to 0.1 wt % and less than or equal to 5.0 wt % sweetener.

16. The dentifrice formulation of claim 9, wherein the dentifrice formulation comprises a whitening agent selected from the group consisting of titanium dioxide, hydrogen peroxide, sodium tripolyphosphates, and mixtures thereof.

17. The dentifrice formulation of claim 16, wherein the dentifrice formulation comprises greater than or equal to 0.1 wt % and less than or equal to 2.0 wt % whitening agents.

18. The dentifrice formulation of claim 9, wherein the dentifrice formulation comprises an abrasive selected from the group consisting of silica, zinc orthophosphate, sodium bicarbonate, alumina, calcium carbonate, calcium pyrophosphate, and mixtures thereof.

19. The dentifrice formulation of claim 18, wherein the dentifrice formulation comprises greater than or equal to 0.1 wt % and less than or equal to 20.0 wt % abrasives.

20. The dentifrice formulation of claim 1, wherein the dentifrice formulation comprises:
greater than or equal to 2 wt % and less than or equal to 15 wt % bioactive glass;
greater than or equal to 30 wt % and less than or equal to 45 wt % solvent;
greater than or equal to 40 wt % and less than or equal to 50 wt % humectant;
greater than or equal to 0.2 wt % and less than or equal to 1.0 wt % fluoride ions;
greater than or equal to 0.5 wt % and less than or equal to 1.5 wt % flavoring;
greater than or equal to 0.5 wt % and less than or equal to 1.5 wt % surfactant;
greater than or equal to 0.1 wt % and less than or equal to 0.5 wt % sweetener;
greater than or equal to 2 wt % and less than or equal to 8 wt % abrasives; and
greater than or equal to 0.1 wt % and less than or equal to 0.5 wt % whitening agents.
